(19) United States
(12) Patent Application Publication
(10) Pub. No.: US 2022/0378377 A1
(43) Pub. Date: Dec. 1, 2022

(54) AUGMENTED ARTIFICIAL INTELLIGENCE SYSTEM AND METHODS FOR PHYSIOLOGICAL DATA PROCESSING

(71) Applicant: Strados Labs, Inc., Philadelphia, PA (US)

(72) Inventors: Yu Kan Au, West Hartford, CT (US); Richard Michael Powers, Smyrna, GA (US); Jason Mark Kroh, Locust Grove, GA (US); Nicholas Shane Delmonico, Wilmington, DE (US); Tanziyah Muqeem, Cary, NC (US)

(73) Assignee: Strados Labs, Inc., Philadelphia, PA (US)

(21) Appl. No.: 17/705,585

(22) Filed: Mar. 28, 2022

Related U.S. Application Data
(60) Provisional application No. 63/194,333, filed on May 28, 2021.

(51) Int. Cl. A61B 5/00; A61B 5/08; G06N 20/00; G16H 10/60

(52) U.S. Cl. CPC A61B 5/7203; A61B 5/7267; A61B 5/08; A61B 5/6801; G06N 20/00; G16H 10/60; A61B 2560/0242

(57) ABSTRACT

In various embodiments, a system for cleaning, marking, and/or interpreting physiological data is disclosed. The system includes a memory having instructions stored thereon, and a processor configured to read the instructions to: receive a training data set comprising physiological data including labeled events corresponding to a predetermined portion of the physiological data, generate a trained artificial intelligence (AI) model configured to identify events within device data, and identify at least one physiological event within a target device data set based on the trained AI model. The trained AI model is generated using an iterative training process based on the training data set.

### CROSS-REFERENCE TO RELATED APPLICATIONS

This application claims benefit under 35 U.S.C. 119 to U.S. Provisional Patent Appl. Ser. No. 63/194,333, filed May 28, 2021, entitled “Augmented Artificial Intelligence System and Methods for Physiological Data Processing,” the disclosure of which is incorporated herein in its entirety.

### BAYH-DOLE ACT STATEMENT

This invention was made with government support under Award ID: 2014713 awarded by the NSF. The government has certain rights in the invention.

### TECHNICAL FIELD

This application relates generally to machine learning and, more particularly, to preparation of physiological data for machine learning.

### BACKGROUND

Recent developments in computer processing and wearable technologies have led to ever increasing amounts of physiological data for processing and interpretation. Numerous machine learning methods have been developed to process physiological data. However, physiological signals acquired in the clinical setting are often complex and include numerous artifacts. This poses challenges to cleaning and marking physiological data to prepare a dataset for analysis, incorporation of new data, and deployment of AI systems in a complex clinical environment in which physiological signals serve as inputs to AI systems.

Human cleaning and marking of data are labor intensive, time consuming, and generally require evaluation of physiological data by human experts. However, human experts are expensive and may not be readily available for the time-consuming task of manually “cleaning” and “marking” data.

### SUMMARY

In various embodiments, a system is disclosed. The system includes a memory having instructions stored thereon and a processor. The processor is configured to read the instructions to receive a training data set comprising physiological data including labeled events corresponding to a predetermined portion of the physiological data, generate a trained artificial intelligence (AI) model configured to identify events within device data, and identify at least one physiological event within a target device data set based on the trained AI model. The trained AI model is generated using an iterative training process based on the training data set.

In various embodiments, an artificial intelligence (AI)-enabled environment is disclosed. The AI-enabled environment includes a first staged processing layer configured to receive device data. The first staged processing layer includes a trained AI model configured to identify at least one physiological event within the device data and the trained AI model is generated based on a training data set comprising physiological data including labeled events corresponding to a predetermined portion of the physiological data. The AI-enabled environment further includes a second staged processing layer. The second staged processing layer is configured to receive first modified device data comprising a portion of the device data. The AI-enabled environment further includes at least one non-transitory storage configured to store at least one of the device data and the modified device data.

In various embodiments, a computer-implemented method of processing device data is disclosed. The method includes steps of receiving device data from a first device, cleaning the device data to remove at least one artifact using a trained artificial intelligence (AI) model, marking the device data to identify at least one physiological event using the trained AI model, and outputting the cleaned and marked device data for use in an AI training process configured to train a second trained AI model to identify physiological events. The trained AI model is generated based on a training data set comprising physiological data including labeled events corresponding to a predetermined portion of the physiological data.

### BRIEF DESCRIPTION OF THE DRAWINGS

FIG. 1 is a process flow illustrating a computer-implemented method of receiving and preparing physiological data for use in generation of one or more additional machine learning models, in accordance with some embodiments.

FIG. 2 is a process flow illustrating a computer-implemented method of iterative data cleaning and marking to prepare data for generation of one or more additional machine learning models, in accordance with some embodiments.

FIG. 3 is a process flow illustrating a computer-implemented method of validating machine cleaned and marked data, in accordance with some embodiments.

FIG. 4 is a process flow illustrating a computer-implemented method of validating machine cleaned and marked data, in accordance with some embodiments.

FIG. 5 illustrates a user-interface configured to display a spectrographic output of a machine learning model generated using machine cleaned and marked data, in accordance with some embodiments.

FIGS. 6A and 6B illustrate a user-interface configured to display a tracing output of a machine learning model generated using machine cleaned and marked data, in accordance with some embodiments.

FIG. 7A illustrates a user-interface configured to display a portion of an audio spectrogram that has been identified and selected as an input segment for further adaptive noise cancellation and/or processing, in accordance with some embodiments.

FIG. 7B illustrates a user interface including the spectrogram of FIG. 7A after an adaptive noise cancellation AI system has been applied, in accordance with some embodiments.

FIGS. 8A and 8B illustrate a user-interface configured to display raw input data and data processed by a machine learning model generated using machine cleaned and marked data simultaneously, in accordance with some embodiments.

FIG. 9 illustrates a user-interface configured to display pre-marked data segments for review and/or verification by a user, in accordance with some embodiments.

FIG. 10 illustrates a user-interface configured to allow user confirmation of machine learning identified respiratory sounds, in accordance with some embodiments.

FIG. 11 is a process flow illustrating a computer-implemented machine learning method of generating cleaned and marked data for use in generating additional machine learning methods, in accordance with some embodiments.

FIG. 12 is a process flow illustrating a method of generating one or more additional machine-learning algorithms using machine cleaned and marked data, in accordance with some embodiments.

FIG. 13 illustrates a computing environment configured to deploy one or more machine learning algorithms configured to clean and mark input data, in accordance with some embodiments.

FIG. 14 illustrates a process flow for receiving and preparing biometric data using one or more trained machine learning algorithms, in accordance with some embodiments.

FIG. 15 illustrates an AI-enabled cloud environment for cleaning and validating of device data, in accordance with some embodiments.

FIG. 16 illustrates a process flow for processing and storage of device data, in accordance with some embodiments.

FIG. 17 illustrates a computer system configured to implement one or more processes, in accordance with some embodiments.

FIG. 18 illustrates an embodiment of an artificial neural network, in accordance with some embodiments.

FIG. 19 illustrates an exploded view of a wearable device, in accordance with some embodiments.

FIG. 20 illustrates electronic components of the wearable device of FIG. 19, in accordance with some embodiments.

FIG. 21 is a flowchart illustrating a process of collecting and processing physiological data using a wearable device, in accordance with some embodiments.

FIG. 22 illustrates an exemplary sample of the two channels overlaid, in accordance with some embodiments.

FIG. 23 illustrates a result of applying a high pass filter, in accordance with some embodiments.

FIG. 24 illustrates the data of FIG. 23 in the form of a histogram, in accordance with some embodiments.

FIG. 25 illustrates a square of the data of FIG. 23, in accordance with some embodiments.

FIG. 26 illustrates exemplary raw summed data and the data after the low pass filter is applied, in accordance with some embodiments.

FIG. 27 illustrates a plot of a breath, in accordance with some embodiments.

FIG. 28 illustrates a spectrogram based on captured audio data, in accordance with some embodiments.

FIG. 29 is a flowchart illustrating a method of identifying physiological events, in accordance with some embodiments.

FIGS. 30 and 31 are flowcharts illustrating methods of determining the aspiration risk associated with a cough detected using data gathered by wearable device, in accordance with some embodiments.

FIG. 32 is a flowchart illustrating a method of determining the risk associated with a cough, in accordance with some embodiments.

FIG. 33 is a flowchart illustrating a method of determining cough characteristics, in accordance with some embodiments.

FIG. 34 is a flowchart illustrating a method of determining the risk associated with a cough, in accordance with some embodiments.

FIG. 35 is a flowchart illustrating a method for assessing the risk associated with an abnormal respiratory sound, in accordance with some embodiments.

FIG. 36 is a flowchart illustrating a method of characterizing abnormal respiratory sounds, such as adventitious breath sounds, in accordance with some embodiments.

### DETAILED DESCRIPTION

The description of the preferred embodiments is intended to be read in connection with the accompanying drawings, which are to be considered part of the entire written description of this invention. The figures are not necessarily to scale and certain features of the invention may be shown exaggerated in scale or in somewhat schematic form in the interest of clarity and conciseness. Terms concerning data connections, coupling and the like, such as “connected” and “interconnected,” and/or “in signal communication with” refer to a relationship wherein systems or elements are electrically and/or wirelessly connected to one another either directly or indirectly through intervening systems.

In some embodiments, systems and methods related to augmented artificial intelligence (AI) and/or machine learning (ML) systems (collectively referred to herein as AI systems or processes) for processing, cleaning, and preparation of data for use in additional AI processing are disclosed. The disclosed systems and methods provide for training algorithms, iterative improvement systems based on new data, and deployment of AI systems for processing of data, such as data collected by wearable medical monitoring devices. The disclosed augmented AI systems (1) allow “cleaning” and “marking” of received data and (2) rapid validation of the AI cleaned and marked data. The disclosed augmented AI systems efficiently integrate inputs during the data cleaning and marking process.

As used herein, the term physiological data includes, but is not limited to, lung sounds, heart sounds, chest wall motion data, and/or other physiological and/or clinical data. Various embodiments of augmented AI systems are configured to clean, mark, and validate physiological data for machine learning applications, which include but is not limited to improving existing algorithms, developing new algorithms, and/or further analysis of the physiological data.

Additionally, augmented AI systems can include an interface having an adaptive system configured to assist in analyzing the physiological data in conjunction with cleaning, marking, and optionally validating the data. An adaptive system interface may be used to analyze physiological data that has been already been prepared (cleaned, marked, and optionally validated), for example, by one or more automated marking and cleaning processes. The disclosed augmented AI systems may be deployed in any suitable environment, such as, for example, for use in clinical research, patient care, and/or other healthcare settings.

As used herein, the term “cleaning” (and variations thereof including “cleaned,” “clean,” etc.) refers to the processing of a dataset identify, remove, modify, and/or otherwise isolate artifacts within the data. Identifying artifacts may include steps such as annotating, labelling, interpreting, and/or otherwise identifying artifacts. Artifacts include flaws within the data that are caused by equipment, techniques, or conditions during observation and storage of the data. Cleaning of data renders subsequent analysis more reliable and robust, as the subsequent analysis focuses on data of interest without considering artifacts, noise, etc.

As used herein, the term “marking” (and variations thereof including “marked,” “mark,” etc.) refers to the process of annotating, labelling, and/or interpreting the dataset. Each of the annotating, labelling, or interpreting may result in adding a description to patterns identified within the dataset. For example, as used herein, “annotating” data refers to identifying one or more patterns within the data and systematically providing an indicator (i.e., “marking”) the one or more patterns. Exemplary patterns include but are not exclusive of a heartbeat, a wheeze, a cough or a series of coughs, a deep breath, and/or other cardiac and/or respiratory sounds. Annotating may or may not be performed with the aid of additional data, such as, for example, imaging data such as an MRI scan, ultrasound data such as an echocardiogram, vital signs such as blood pressure, laboratory data such as complete blood count, or medical records such as the past medical history, the physician's documented physical exam of the subject from which the physiological data was obtained, motion data, environmental data and/or air quality data (e.g., smog, pollen count level, air pollution index, etc.), location data (such as the location of the patient) and/or any other suitable data type. Additionally, metadata, defined as data that provides information about other data, may also be used during annotation. Exemplary metadata includes, but is not limited to, contextual information associated with the physiological data, such as the fact that a patient was performing deep breathing exercise when wheezes were recorded.

Annotation may be performed using any suitable annotation notation, such as, for example, commonly accepted terminology in physiology, user-defined terminology, AI defined terminology, etc. For example, in some embodiments, a wheeze may be annotated as a wheeze, or it may be annotated as “A1.” In other embodiments, a wheeze may be annotated as “A1,” “A2,” or “A3” based on one or more criteria, such as, for example, whether the wheeze was judged to be loud, normal, or faint, respectively. In some embodiments, multiple “annotations may be applied. The multiple annotations may be applied as alternatives, applied in hierarchies (e.g., layers), and/or using any other suitable organization method. It will be appreciated that annotation, labelling, and/or interpretation, as discussed herein, may be applied to datasets as one or more individual layers to provide for processing, such as for example as one or more hidden layers in a trained machine learning algorithm.

Annotation may be based on pre-specified criteria and/or learned judgement. For example, in various embodiments, a wheeze may be defined by a sound's duration, frequency, power, and/or spectral pattern, defined based on judgement in view of prior experience (e.g., machine learning training based on pre-annotated data identifying a wheeze), and/or annotated as a wheeze only if the reviewed data has a duration that meets a threshold and includes additional criteria identifying the data as a wheeze. Although specific embodiments are discussed herein, it will be appreciated that any suitable criteria may be used to identify events within the dataset.

In some embodiments, the use of the disclosed AI systems allows subtle differences among physiological signals to be systematically captured in a standardized manner and annotated accordingly, which otherwise may not be captured in commonly used descriptions. For example, in some embodiments, both a loud wheeze lasting the entire duration of an exhalation and a faint end-expiratory wheeze may be commonly called a wheeze in a clinical setting. Colloquial descriptions of these two wheezes by physicians may vary. By utilizing trained AI systems (as discussed in greater detail below), each of these sounds may be identified using unique annotations and/or markers allowing for more robust analysis, diagnosis, and/or additional clinical and/or research applications.

As used herein, the terms “labelling” and “interpreting” refer to marking recognized patterns within the data by systematically naming the patterns based on terminology. The terminology may include, but is not limited to, commonly accepted terminology related to the analytical use case in question, system-defined terminology, user-defined terminology, etc. Exemplary use cases include but are not limited to research with specific, custom-made clinical trial endpoints, patient care, or training of a machine learning model. The patterns may or may not be annotated prior to labelling and interpreting of the data.

In some embodiments, separation of annotating, labelling, and interpreting into three different processes, allows the augmented AI data processing system to capture subtle differences in physiological events through annotations, while labelling and interpreting using criteria designed to meet a specific purpose. For example, in some embodiments, event-accurate annotation may be used to uniquely identify different events within physiological data, allowing a system to capture subtle differences important to a specific purpose, while providing labelling and interpretation in a format commonly used within a clinical and/or research setting to allow for rapid and easy application to clinical and/or research settings.

In various embodiments, labelling and/or interpreting may be performed with the aid of additional data. Examples include but are not limited to imaging data such as an MRI scan, ultrasound data such as an echocardiogram, vital signs such as blood pressure, laboratory data such as complete blood count, or medical records such as the past medical history, the physician's documented physical exam of the subject from which the physiological data was obtained, motion data, environmental data and/or air quality data (e.g., smog, pollen count level, air pollution index, etc.), location data (e.g., location of a patient), metadata, and/or any other suitable data type.

As used herein, labelling is distinct from interpreting data in that labelling refers to categorizing manifestation(s) of underlying physiological state(s), while interpreting data refers to categorizing the underlying physiological state itself. For example, lung sounds consistent with wheezes that occur during end-expiration while a subject is in motion consistent with exercising may be labelled as “exercise-induced end-expiratory wheezes.” Concurrently, the lung sounds may be optionally annotated as “end-expiration wheezes,” annotated with a custom-made notation such as “B1,” and/or otherwise annotated by an AI system. Similarly, the associated motion may be annotated as “exercise”, annotated with a custom-made notation such as “E2,” otherwise annotated by the AI system, and/or not annotated.

In some embodiments, the same lung sounds described above may be interpreted as “exercise-induced bronchospasm”. In current clinical settings, interpreting data generally requires training in physiology and synthesizing contextual data to arrive at an interpretation. By applying AI systems configured to interpret the data, expertise and reasoning in physiology can be systematically captured within the marked dataset. It will be appreciated that a dataset may be labelled, interpreted, or both labelled and interpreted, as these two types of “marking” are not exclusive of each other.

In some embodiments, interpretation of data created from multiple data sources and/or data in conjunction (e.g., in context with) data from other sources is made by a trained AI system configured to implement one or more algorithms. In some embodiments, interpretation of the dataset generates a context for the data. The interpretation may be subsequently confirmed and/or corrected. For example, one or more algorithms may interpret data consisting of rapidly diminishing lung sounds and wheezing over two hours, increasing respiratory rate as captured by motion sensors over the same two hours, and a medical history of severe chronic heart failure in the medical record. The interpretation applied to the data by the AI system may be a “flash pulmonary edema” event. This interpretation of two-hour's worth of data may then be verified or corrected, for example, by an AI system specifically trained to identify pulmonary edema events and/or by a clinician. Subsequently, the interpretation may further be validated as pulmonary edema. The dataset, having been marked and “validated”, may then be used for additional machine-learning applications, such as, for example, further training of detection and/or diagnostic AI systems. In some embodiments, validation includes a process of affirming that the physiological data was “cleaned” and “marked.”

In yet another example, one or more algorithms may provide an interpretation of data related to an event that may have occurred prior to collection of the data being interpreted, simultaneous with the data that is being interpreted, and/or that may occur at some point after the time at which the data are collected. For example, one or more algorithms and/or trained AI systems may interpret increasing heart rate and increasing amplitude of wheezes over four hours as an “event” predictive of flash pulmonary edema, but the actual event, e.g., the flash pulmonary edema, may have occurred two days before the time at which the interpreted data was collected. In yet another example, one or more algorithms and/or trained AI systems may “interpret” decreasing heart rate and decreasing amplitude of wheezes over four hours as an event suggestive of a flash pulmonary edema, but the actual event, e.g., the flash pulmonary edema, may not occur until two days after the time at which the interpreted data was collected. Interpretation of data by one or more algorithms and/or trained AI systems using one or more data sources can provide marking of events concurrent to a clinical event that happens at the same time as the marked event, predictive of a clinical event at a certain future time point from the time when the data is collected, and/or suggestive of a clinical event at a certain past time point before the time when the data is collected. The data used for interpretation may be from the same source or multiple sources, and may be from the same point in time or different points in time.

In some embodiments, the interpretation of data correlated with event(s) which may occur at a certain point in time before time at which the data was collected, after the collection of the data, and/or concurrently with collection of the data, enables the construction of databases based on which prospective and/or retrospective clinical studies can be performed to arrive at clinically validated prediction tools, such as trained AI systems configured to identify and/or predict physiological events. In some embodiments, datasets and event identification may be validated prior to inclusion in a database.

In some embodiments, an input dataset may include, but is not limited to, physiological data such as thoracic and abdominal sounds including lung sounds, heart sounds, and/or other sounds emanating from structures of the thoracic and abdominal cavities (such as, for example, bowel sounds or sounds generated by movements of the diaphragm). Sounds may originate from normal physiology and/or disease processes including, but not limited to, a diseased heart valve, bleeding in the abdomen, fluid in the lungs, obstructions of the bowels, and/or other physiological and/or diseased processes. Sounds may be in the audible range and/or in an inaudible range including, but not limited to, ultrasonic frequencies. In some embodiments, sound may be acquired from any suitable source, such as, for example, a wearable device, a contact microphone, a condenser microphone, and/or other sound acquisition devices such as an electronic fabric with sound acquiring function. Sound may be acquired with or without skin contact and may be captured continuously and/or periodically. Suitable wearable devices are disclosed in U.S. Pat. Appl. Publ. No. 2018/01777432 and International Pat. Appl. Pub. No. WO2019241674A1, the disclosure of each of which is incorporated herein by reference in their entireties.

In some embodiments, an input dataset includes, but is not limited to, physiological data such as body motion, such as, for example, chest wall motion, abdominal wall motion, whole body motion, and/or any other suitable motion. Body motion may include linear and/or angular motion and may be acquired by one or more devices with or without skin contact. Exemplary devices include, but are not limited to, wearables, fabrics, elastic bands, accelerometers, gyroscopes, magnetometers, video cameras, infrared cameras, technologies based on Doppler techniques, and/or ultrasound technologies that can sense motion. Motion data may be continuous or fragmented (e.g., asynchronous or non-continuous) and may be acquired from multiple sources and integrated for further analysis.

In some embodiments, an input dataset includes, but is not limited to, additional physiological data obtained from various sources including, but not limited to, demographics, medical records, oxygenation level, carbon dioxide level, electrocardiogram, electroencephalogram, laboratory results, vital signs, radiographic data (including echocardiogram and other ultrasound imaging), nursing assessments, patient-reported data, wearable data, environmental data, ambient temperature, ambient humidity, geographic location, and/or an associated disease prevalence.

In some embodiment, input data may be modified by one or more subject behaviors, environment conditions, device configurations, and/or other factors that may affect the acquisition and characteristics of the input data. In some embodiments, a condition which leads to input data modification is defined as an “input modifier.” Input modifiers may be captured as metadata, and may aid in the selection of staged processing pathway based on the characteristics of the input modifier.

In various embodiments, subject behaviors include those that are spontaneous (initiated by the patient without being directed to do so) and/or those that are directed by an entity other than the subject, such as a caregiver, a clinician, an automated system configured to implement one or more diagnostic algorithms, etc. In some embodiments, an automated system may provide instructions to a subject via one or more human-computer interfaces, such as, for example, via a graphical user interface, audio systems, visual systems, etc.

In some embodiments, a subject may be directed to perform one or more actions or activities for diagnostic purposes. For example, a subject may be instructed to “take a deep breath” or perform other breathing exercises to identify a respiratory sound that may be modified by a deep breath (e.g., becoming louder, transitioning from not containing a wheeze to containing a wheeze, etc.). The subject may be instructed to perform the breathing action by, for example, an application on a computerized device, such as a smartphone, may direct the patient to take deep breaths, a clinician via a video call or phone call, and/or via any other suitable interface. In some embodiments, metadata regarding the type of modifier of the input data, such as being associated with taking deep breaths, is associated with the input data which it modifies. The input data may be obtained by one or more devices, such as a wearable device, to capture physiological data for use in further analysis and/or diagnostics.

As another example, in some embodiments, a subject may be directed to cough. The cough is a respiratory sound that is included in the input data and the direction to cough is an input modifier that is associated with the input data. In some embodiments, data annotation, validation, interpretation, and/or the staged processing of the input data utilizes the input conditions as an input, as described elsewhere herein. In some embodiments, input data processing and/or use of input conditions may be limited a predetermined time period, such as, for example, three minutes before and/or three minutes after a directed cough event. In some embodiments, a subject with airway secretions may have one or more conditions, such as rhonchi, which may be cleared after a cough or other event, evaluation of lung sounds pre and post directed event may be helpful to a clinician and/or an AI system as a diagnostic and/or therapeutic maneuver. In some embodiments, staged processing of input data tailored for a specific application based on input modifier information makes data processing more efficient and aids in data interpretation.

In some embodiments, input modifiers include environmental conditions, such as, for example, temperature, humidity, ambient noise, etc. In some embodiments, ambient noise above a predetermined threshold may be used as an input modifier such that input data associated with the ambient noise modifier goes through a different processing pathway during staged processing to provide optimal processing (for example, to include additional filtering, noise cancellation, etc.). In another example, input data associated with ambient noise above a certain threshold is annotated, validated, and interpreted using a single pathway but may be selectively excluded in applications that require input data from an environment with noise below a certain said threshold.

In some embodiments, ambient temperature are included as an input modifier. For example, extremely cold or hot weather may affect a frequency response of materials used in data acquisition devices. To compensate for differences in data acquisition, the processing of input data with an input modifier of a certain ambient temperature may be different from the processing of the same type of input data with an input modifier of a different ambient temperature, such that device frequency response difference may be taken into account during data processing.

In some embodiments, one or more devices characteristics are included as an input modifier. For example, a wearable device may vibrate on a body surface such that the motion of the wearable device may mimic that of percussion by a physician. The audio input data captured during device vibration may be associated with a device vibration input modifier such that annotation, validation, and interpretation of data would be different compared to the same type of audio input data not associated with this particular input modifier. As another example, two wearable devices may be placed on different locations of the thorax to capture lung sounds. The configuration of two wearable devices in two predetermined locations may be an input modifier such that the input data from the two devices are siphoned to a specific stage processing pathway that allows the localization of a disease process based on difference in the audio signals from the two steams of input data.

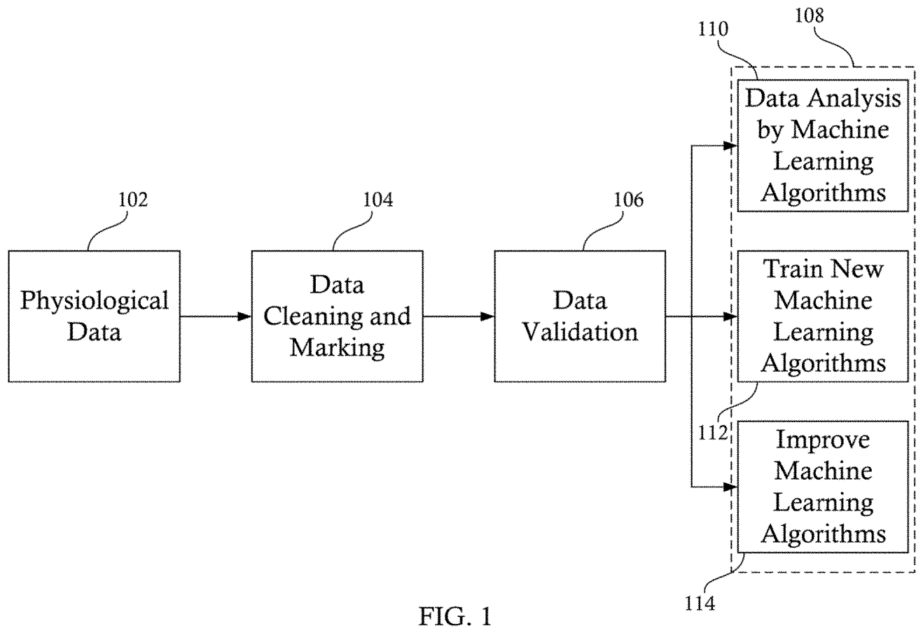

FIG. 1 is a process flow 100 illustrating various steps of a computer-implemented method of receiving and preparing physiological data for use in generation of one or more additional machine learning models, in accordance with some embodiments. Physiological data 102 may be received from one or more sources. For example, physiological data 102 may be received from one or more wearable devices, one or more mobile computing devices, one or more databases, and/or any other suitable source. The physiological data 102 may include data from a single subject and/or data from multiple subjects.

In various embodiments, the physiological data 102 may be cleaned, marked 104 and/or validated 106. Although embodiments are illustrating including a cleaning and marking step 104, it will be appreciated that data may be marked with or without cleaning and that cleaning and marking may be performed as separate steps. Current systems include validation that is generally performed by a human who is an expert in the physiological data that is being processed. In the disclosed AI systems, the process of validation is performed, at least partially, by the AI system. In some embodiments, validation is configured to ensure quality assurance of the annotation process and may include, for example, a sanity check that ensures the cleaned and marked data makes sense in the context(s) specific to the identified application(s). Validation of the same cleaned and marked data may yield different results depending on, for example, associated contextual metadata and/or other input modifiers.

The cleaned, marked, and/or validated data may be used for one or more additional processes 108, such as, for example, used as input to one or more additional AI systems or models 110 for analysis (including, but not limited to, filtering and/or other mathematical processing (such as Kalman filtering)), used to improve existing machine learning models 112, and/or used as a training data set to train new algorithms 114.

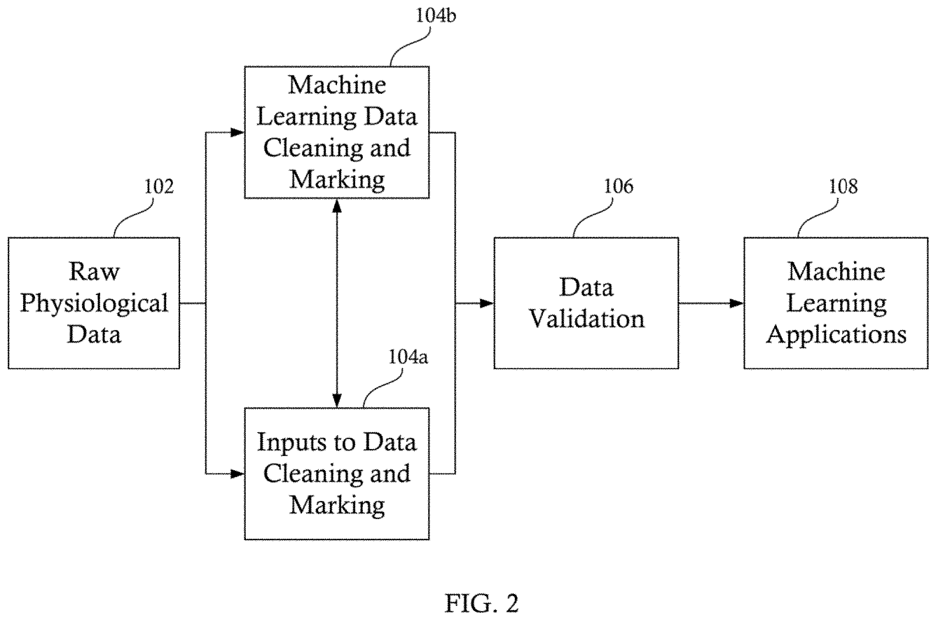

FIG. 2 illustrates a process flow 100a illustrating various steps of a computer-implemented method of iterative data cleaning and marking to prepare data for generation of one or more additional machine learning models, in accordance with some embodiments. The process flow 150 is similar to the process flow 100 of FIG. 1, and similar description is not repeated herein. As shown in FIG. 2, in some embodiments, the cleaning and mark process 104 may be divided into an iterative process including AI cleaning and marking 104b using a pre-trained AI model and selection of inputs for data cleaning and marking 104a based on an output of a previous iteration of the trained AI model. In some embodiments, the iterative process of selecting and cleaning/marking data may be performed a predetermined number of times to ensure that the physiological data 102 has been properly cleaned and/or marked.

In some embodiments, the trained AI model is configured to annotate, label, and/or interpret input data. For example, in some embodiments, the trained AI model is configured to clean input data to remove noise and other artifacts and is further configured to mark a set of events within a predetermined area of interest, such as, for example, respiratory events, cardiac events, etc. The input data may be annotated, labeled, and/or interpreted using a standard lexicon, custom lexicon, and/or use-case specific terminologies.

In some embodiments, external or environment sounds may be marked and/or interpreted for removal or isolation during further processing. For example, in some embodiments, speech is marked for optional subsequent removal to ensure privacy of the subjects from whom the physiological data were obtained and/or privacy of third parties (e.g., persons located within recording distance of the device). Speech from the subject from whom the physiological data were obtained may be differentiated from the speech originating from person or persons in the vicinity of the device but whose speech is not the sound of interest. Speech from person or persons in the vicinity of the device that were captured may undergo further processing with optional removal to ensure the privacy of person or persons in the vicinity of the device from whose physiological data were not the data of interest.

In some embodiments, respiratory sounds such as coughs or loud wheezes originating from person or persons in the vicinity of the device are differentiated from respiratory sounds originating from the subject of interest from whom physiological data were obtained. In this exemplary configuration, the respiratory sound or speech resonance frequency, amplitude, motion data, and/or other acoustic properties captured by a device may be used to differentiate whether speech or respiratory sounds originated from the subject of interest versus person or persons who are in the vicinity of the device but who are not the intended target of physiological data collection. In one embodiment, soundwave paths from an external source will travel through different layers of materials than the soundwave path of an internal signal.

For example, the signal path of an external sound may predominately travel through a hard enclosure and cause vibrations on the hard surface of a PCB to the microphone which will pass higher frequency content more readily than lower frequency content. In contrast, the signal path of an internal sound travels through tissue, for example, to a diaphragm and bell structure to a column of air to the microphone which will pass low frequency content more readily than high frequency content. The energy of the frequency content of each noise can be measured and compared. In some embodiments, if the sound originated internally, the data will include a larger percentage of low energy frequency content than high frequency content. If the sound originated externally there will be more high frequency content. Additional analysis, such as, for example, analyzing energy in the harmonics may be used. For external sounds, the energy content of the harmonics will increase from the lower harmonic to the higher harmonic, whereas, for internal sounds, the energy content of the harmonics will decrease from the lower harmonic to the higher harmonic. In one embodiment, the slope of a line made up of the peaks of a Fast Fourier Transform (FFT) can be used to detect whether a sound originated externally or internally.

In some embodiments, a calibration process may be performed prior to and/or in conjunction with capturing of the physiological data and/or training of the AI model. For example, in some embodiments, a user wearing a wearable device configured to obtain physiological data may be prompted to speak a particular pattern or set of works. A trained model may be configured to compare a frequency response of the spoken sample with harmonics to identify certain markers and/or other identifiers for speech data.

In some embodiments, audio characteristics (e.g., energy content in harmonics, frequency content, spectral content, etc.) of the device data are used to determine if a wearable device has adequate contact with the body. The audio characteristics of an internal sound captured by a wearable device with adequate contact with the body differ from the audio characteristics of an internal sound captured by a wearable device without adequate contact with the body. Soundwave paths of an internal sound captured by a wearable device having adequate contact are different from soundwave path of internal sounds captured by a wearable device having inadequate contact. When there is inadequate contact between the wearable device and the body, the internal sound may travel through air between the body and the device, and the amount of air will vary depending on the level of contact. Additionally, if there is inadequate contact between the wearable device and the body, internal sounds travel through skin and subcutaneous tissues having less tension and/or travel through a wearable device surface that has less tension. In both cases, the audio characteristics of the signal change due to changes in the vibrational properties of the substances along the soundwave path. In some embodiments, the audio characteristics of an internal signal are used to assess whether a wearable device has adequate contact with the body. Although specific embodiments are discussed herein, it will be appreciated that any suitable cleaning, marking, and/or interpretation mechanisms may be used to remove and/or isolate undesired data from desired data.

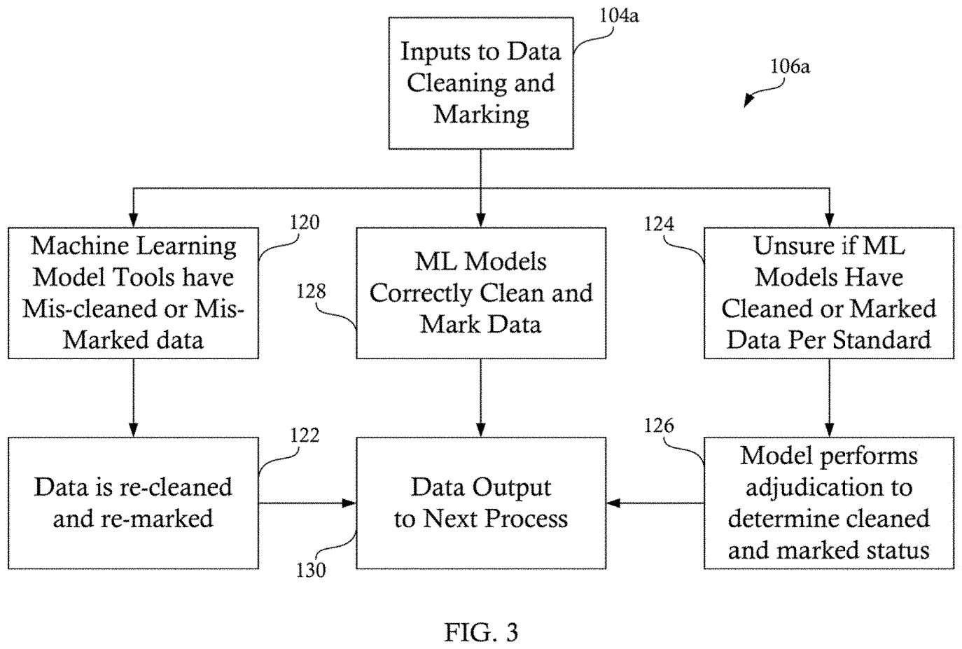

FIG. 3 is a process flow 106a illustrating a computer-implemented method of validating machine cleaned and marked data, in accordance with some embodiments. As illustrated in FIG. 2, an AI cleaning and marking model 104b generates intermediate marking and classifications that are used as further inputs 104a to the AI cleaning and marking model 104b. FIG. 3 illustrates a process of validating the generated intermediate inputs, in accordance with some embodiments. During validation, the generated inputs may be processed to identify mis-cleaned and/or mismarked data 120 and/or cleaned/marked data outside of one or more confidence thresholds 124.

If an intermediate input is identified as being mis-cleaned and/or mismarked, the data that generated the intermediate input is re-cleaned and/or re-marked to generate a new intermediate input. For example, in some embodiments, a marking, such as a “couch” designation, may include upper and/or lower thresholds for one or more characteristics, such as frequency, power, etc. If one or more of the parameters falls outside of the upper and/or lower thresholds, the data may be identified by a trained model as being “mismarked,” which may be a result of incorrect cleaning (e.g., portions of the data removed that should have been kept, portions not discarded that should have been removed, etc.). When data is identified as being mis-cleaned and/or mismarked, the data may be re-cleaned and/or re-marked. Additional validation may be performed to re-validated the newly cleaned and marked data before using the data for machine learning applications.

Similarly, in some embodiments, the AI system 104b may generate an intermediate input having a confidence threshold below a predetermined level. If marking confidence is below a predetermined threshold, an adjudication process 126 may be applied to determine whether the cleaning and marking of the data was accurate. For example, in some embodiments, an adjudication process 126 may include comparison of the marked data to previously marked data to confirm the marking classification. As another example, in some embodiments, the adjudication process 126 may apply a different trained AI model configured to remark and/or verify the marking of the initial trained AI model. Although specific embodiments are discussed herein, it will be appreciated that any suitable verification process may be employed to verify marking and/or cleaning of the input data.

As illustrated in FIG. 3, if the verification process 106a determines the AI system correctly cleaned and marked the data 128, the marked and cleaned data may be provided as an output 130 for use in one or more additional processes, such as, for example, processing by one or more additional trained AI models configured to perform additional clinical, research, and/or other tasks, such as, for example, an AI system configured to perform disease diagnostics based on the marked and cleaned data.

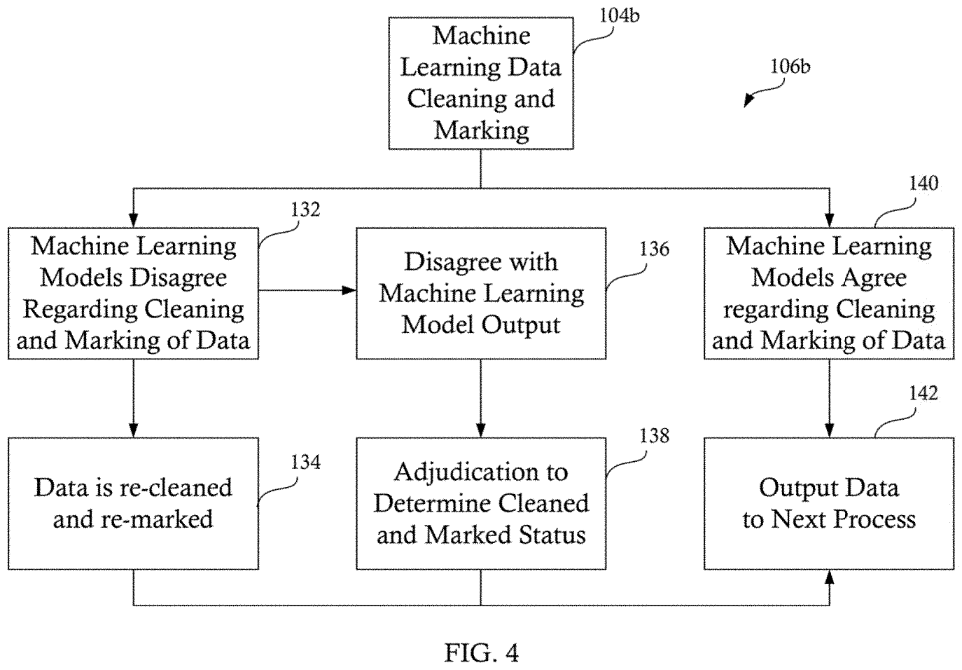

FIG. 4 is a process flow 106b illustrating a computer-implemented method of validating machine cleaned and marked data based on the output of a trained AI system, in accordance with some embodiments. In some embodiments, data may be cleaned, marked, and/or otherwise processed by multiple trained AI systems. During validation 106b, the results of each of the trained AI systems may be compared. If two or more trained AI systems (or two or more applications of the same AI system) disagree, the data may be re-cleaned and/or re-marked 134 by one or more trained AI systems, such as the previously applied AI systems and/or a different AI system. The re-processed data may undergo subsequent validation to determine the accuracy of the re-marking and/or re-cleaning.

In some embodiments, when there is disagreement between the machine learning outputs 136, an adjudication process 138 may be applied to determine the correct marking and/or cleaning of the subject data. The basis of the disagreement may be evaluated, for example, by one or more additionally trained AI models. The adjudication process 138 is configured to determine which of the AI outputs are most likely correct and selects that output as the output data. In some embodiments, the output of the adjudication process 138 may be used for further training and/or refinement of the trained AI models.

In some embodiments, the data “cleaning” and “marking” process(es) are fully automated. When the probability of correct classification (e.g., as determined, for example, by one or more trained machine learning algorithms) falls below a certain threshold, an alert mechanism may be configured to trigger additional review of the data. The additional review may be performed using any suitable mechanism, such as, for example, automated and/or manual review.

With reference again to FIG. 2, in some embodiments, a trained AI system configured to clean and/or mark an input data set may be configured to utilize a traditional algorithm to perform initial cleaning and/or marking of data and subsequently applies a trained model (e.g., one or more trained layers) to further mark the data. For example, a portion of the input data may be initially marked as a “wheeze” based on analysis of one or more characteristics, such as, for example a start and stop time of the portion of the input data in conjunction one or more frequency occurrences within the portion of the data. After the initial (e.g., simple) classification, a trained AI system (e.g., a trained machine learning model) is applied to the “wheeze” to perform additional classification. For example, in some embodiments, the trained AI system is configured to perform a more detailed wheeze, marking the initially identified wheeze as a specific type of wheeze, e.g., a “B2 wheeze.” The trained AI system may be configured to utilize any suitable properties of the input data, such as, for example, duration, frequency, timing, etc. Thus, algorithms aid in more precise marking of input data. Criteria specific to a use case may be used to further mark the input data so as to best prepare the data for further analysis in a manner that is most suitable for that specific use case.

With reference again to FIG. 2, in some embodiments, the input data may include body sounds and body motion data recorded by one or more devices, such as, for example, a wearable device. In some embodiments, the trained AI model 104b is configured to clean and mark both body sound data and motion data. The motion data may include, but is not limited to, acceleration data, velocity data, displacement data, and/or any other suitable form of motion data. In some embodiments, the length of each sound or motion data event segment is defined and each defined sound or motion data event segment is marked.

In some embodiments, conversion between data types may be used to aid in cleaning and/or marking of the data. For example, in some embodiments, identification of overlapping sound and motion data segments may allow comparison and/or combination of motion and sound data points during cleaning, marking, and/or interpretation. The trained AI model 104b may be configured to utilize any suitable data input, such as, for example, sound input, motion input, other physiological and/or environmental data inputs, etc. for use in cleaning, marking, and/or interpretation of the physiological data 102.

In some embodiments, input data may be displayed visually and/or communicated via audio without signal processing or at various stages of signal processing, to provide validation and/or assurance to a user regarding the cleaning, marking, and/or interpretation performed by the trained AI model 104b. Multiple sources of data may be communicated simultaneously. Data may be displayed in the time domain, the frequency domain, and/or any other suitable domain. Audio may be communicated in real time, in a time-condensed format, and/or at other time scales. Visual and audio data may be displayed in raw form or after processing with filters, or after machine learning processing to identify key information to be communicated. Color schemes and audio markers are exemplary schemes that may be used to identify key information clusters for processing.

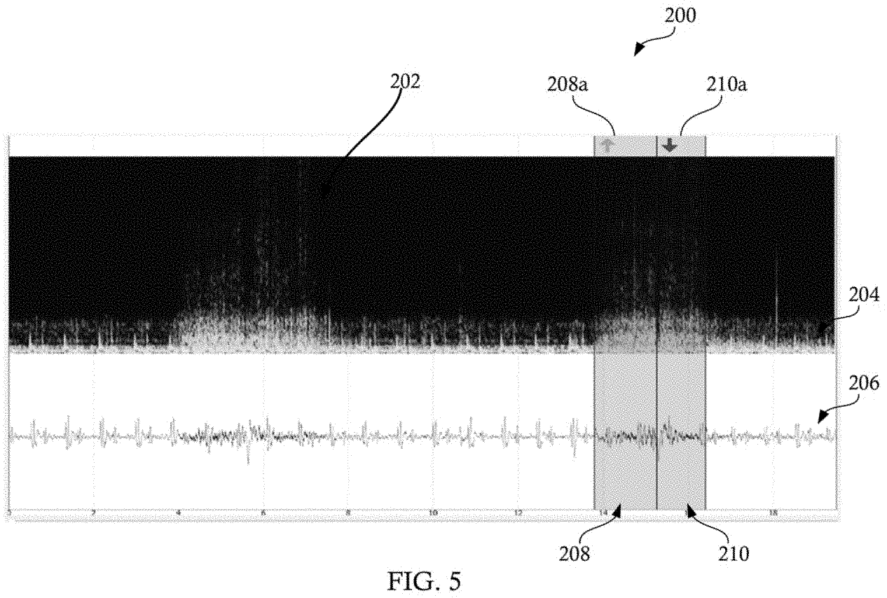

FIG. 5 illustrates a partial user-interface 200 configured to display a spectrographic output of a machine learning model generated using machine cleaned and marked data, in accordance with some embodiments. The user-interface 200 includes a spectrogram 202 of lung audio data, a spectrogram 204 of heart audio data, and a combined waveform 206 illustrated as amplitude vs. time. In some embodiments, sound data, such as lung sound data 202 and/or heart sound data 204, may be displayed visually as spectrograms. Several frequency filters including, but not limited to, low pass, high pass, notch, and/or manually-set frequency filters may be available. In some embodiments, abnormal lung sounds are identified using machine learning methods and are highlighted (See FIGS. 5,9).

The user-interface 200 may further include AI-generated markers indicating marked data identified by the trained AI system 104b. For example, in the illustrated embodiment, the user-interface 200 includes a first AI-generated marker 208 indicating an AI-identified inhalation and a second AI-generated marker 210 indicating an AI-identified exhalation. In some embodiments, additional markers 208a, 210a may be configured to provide additional context to the AI-generated markers 208, 210.

In some embodiments, the trained models, such as trained AI model 104b, are configured used to mark events, such as abnormal lung sounds, and generate visual indications of the marking, such as highlighting, natural language, images, and/or any other suitable indicators. In some embodiments, a confidence level associated with each marked event may be displayed, for example, as a percentage or a range of percentages. Marked events having a machine learning output confidence level below a predetermined threshold may be highlighted in a different color than events having a confidence level above the predetermined (or other) threshold. In embodiments including physiological data having recorded lung or abdominal sounds, the highlighted and marked events may include, but are not limited to, abnormal respiratory sounds, normal respiratory sounds, respiratory phases (e.g., inspiration and expiration), artifacts, environmental sounds, and/or any other suitable sound events.

In some embodiments, heart sounds 204 may be visually displayed and/or marked. Abnormal and/or normal heart sounds may be marked and indicated using words, highlighting, tags, etc. The marking may include an estimated accuracy of identification by the trained AI model, as discussed above.

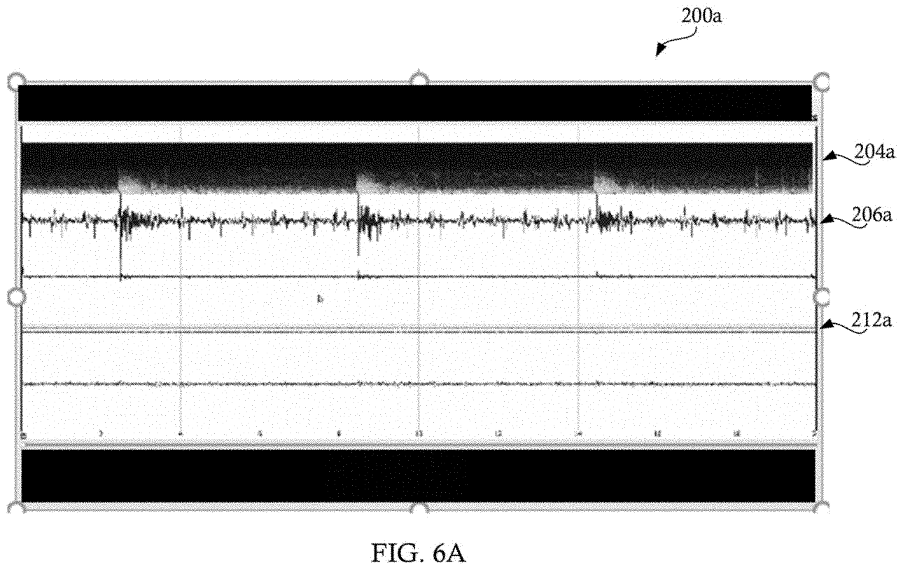

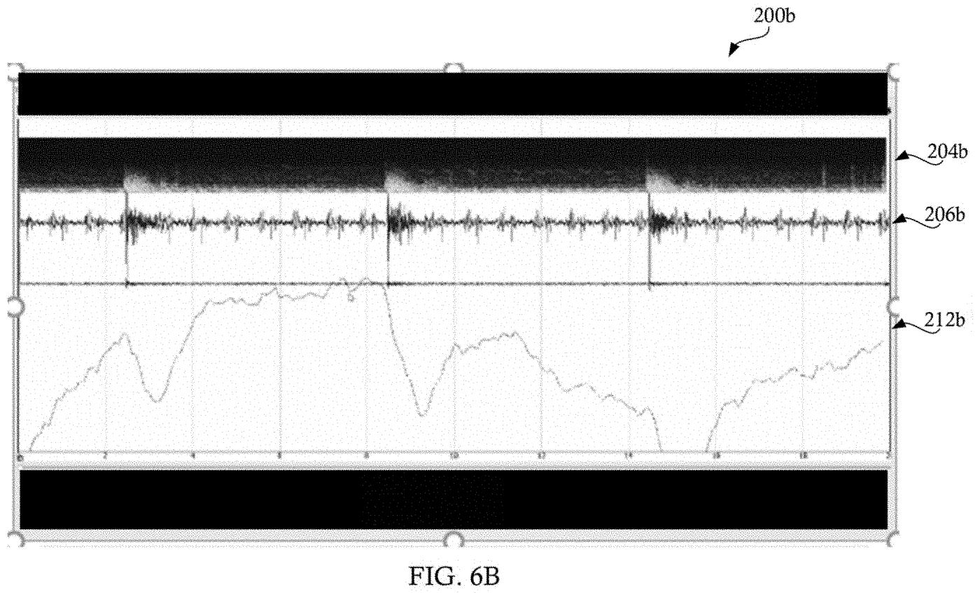

FIGS. 6A and 6B illustrate user interfaces 200a, 200b including audio tracings 206a, 206b illustrating an audio signal in the amplitude and time domains, spectrograms 204a, 204b of the audio tracing 206a, 206b, and motion data tracings 212a, 212b. The user-interfaces 200a, 200b are similar to the user interface 200 discussed above, and similar description is not repeated herein. The motion data tracings 212a, 212b may be generated based on any suitable motion data, such as, for example, chest wall motion data. The motion tracings 212a, 212b may be generated based on raw motion data and/or processed motion data and may be configured to displaying position, velocity, acceleration, and/or any other suitable parameter. In some embodiments, a Kalman filter is used to combine multiple types of sensor data for display as a single tracing.

In some embodiments, motion data is cleaned and marked by the trained AI system 104b and events, such as inspiration, expiration, and/or coughs, are highlighted, marked with words, and/or tagged with an estimated accuracy of marking on the user interface 200-200b.

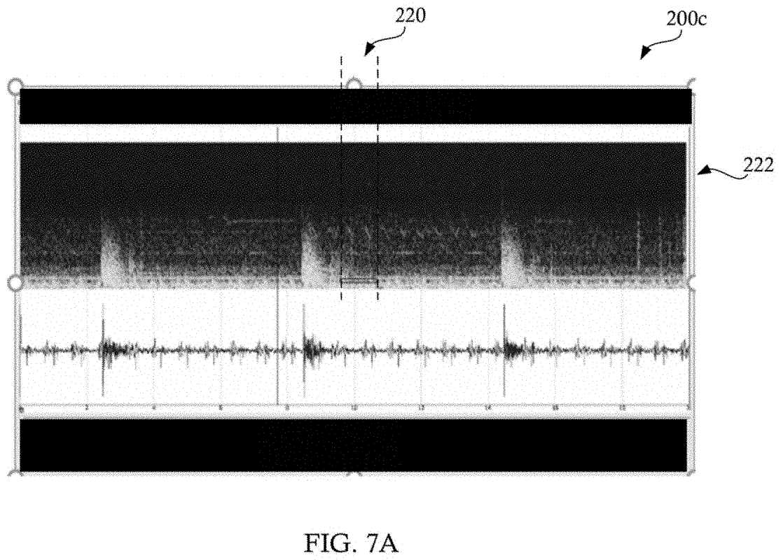

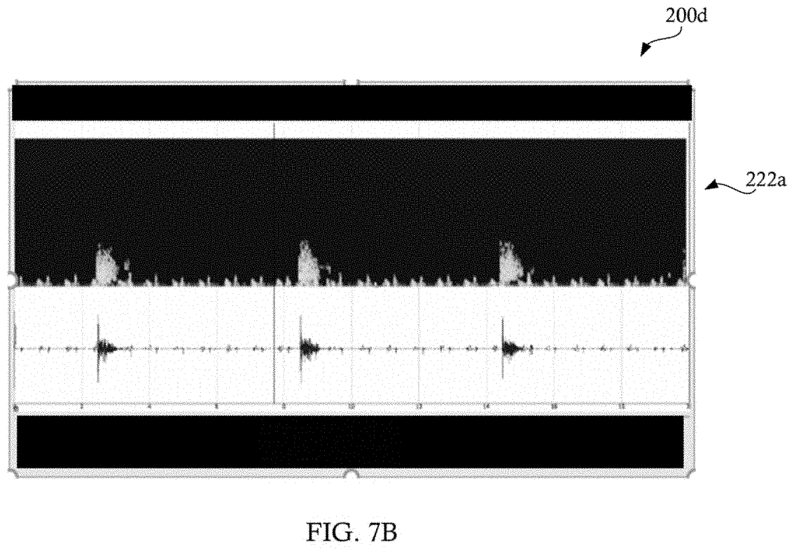

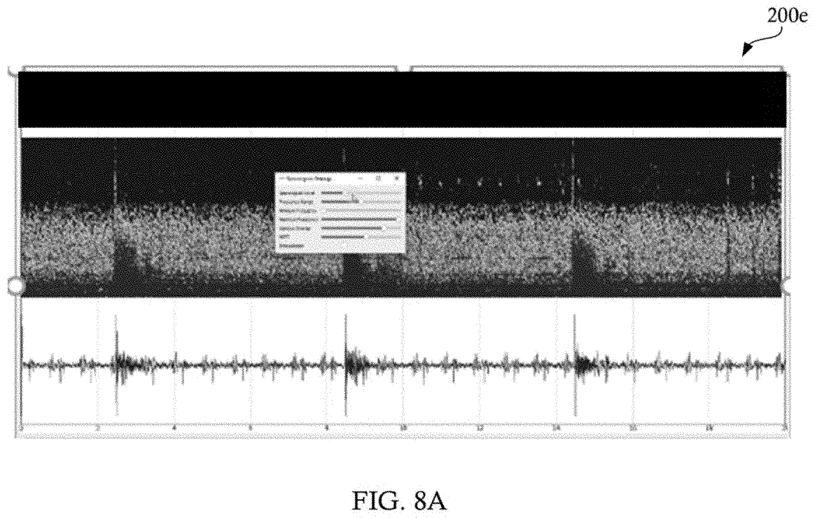

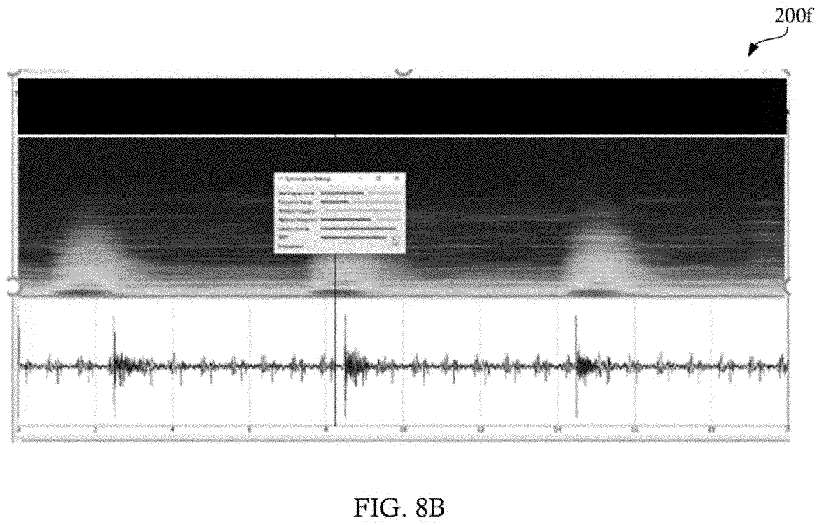

FIGS. 7A-8B illustrate additional embodiments of a user-interface configured to provide visual representations of various data elements, in accordance with various embodiments. For example, FIG. 7A illustrates a user-interface 200c including a portion 220 of an audio spectrogram 222 that has been identified and selected as an input segment for further adaptive noise cancellation and/or processing. The selected input segment 220 and the spectrogram 222 are provided to a trained AI model for further processing. FIG. 7B illustrates a user interface 200d including the spectrogram 222a after an adaptive noise cancellation AI system has been applied. FIGS. 8A and 8B similar include user-interfaces 200e, 200f that allow for display and/or manipulation of physiological data such as lung sounds, heart sounds, motion data, etc., either in raw format or in processed form.

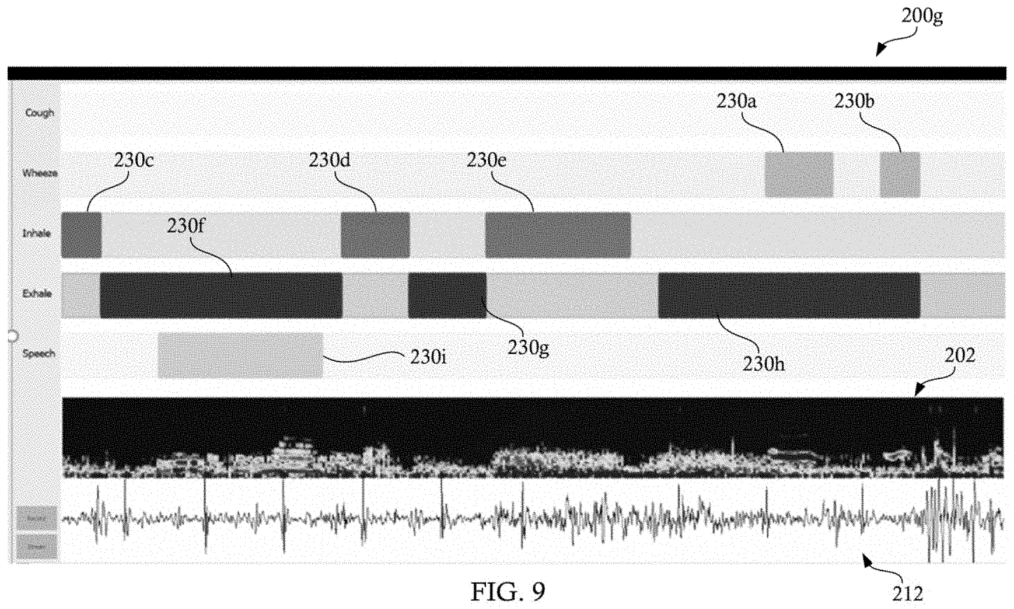

In some embodiments, a user may interact with a user interface to verify, overwrite, and/or otherwise interact with generated data markings. For example, FIG. 9 illustrates a user-interface 200g configured to display pre-marked data segments 230a-230i corresponding to AI-marked sounds 232a-232e for review and/or verification by a user, in accordance with some embodiments. In addition to highlighting segments 230a-230i identified by the trained AI system 104b, the user-interface 200g may include additional data, such as, for example, an audio spectrogram 202 and/or an audio tracing 212.

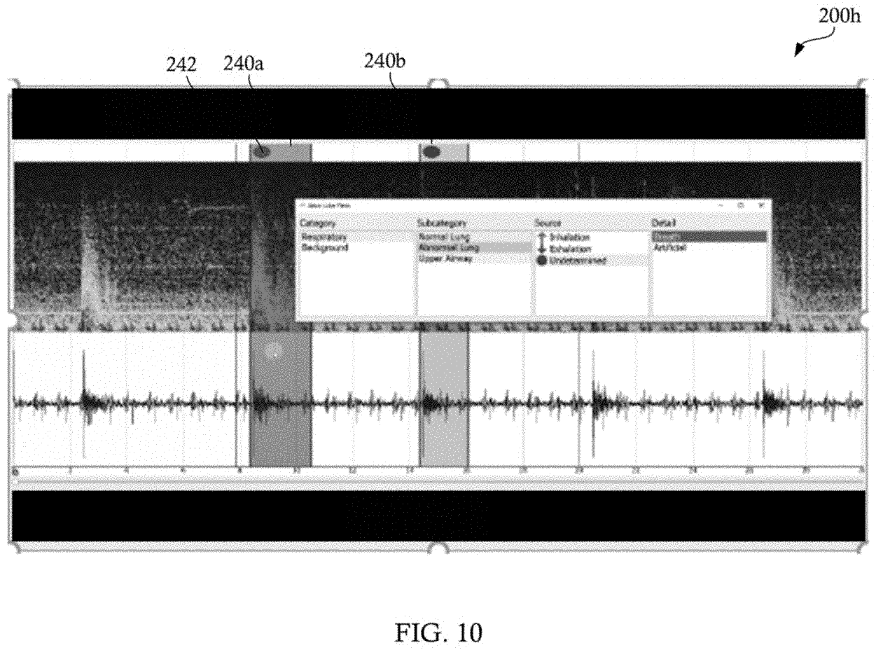

Similarly, FIG. 10 illustrates a user-interface 200h configured to allow user confirmation of machine learning identified respiratory sounds, in accordance with some embodiments. The user-interface 200h includes a plurality of highlighted segments 240a-240b including a visual indicator 242 corresponding to the classification (e.g., marking) applied by the trained AI model 104b. The user-interface 200h may further include one or more inputs 242 to allow a user to re-mark and/or re-interpret the AI-marked data, as discussed in greater detail below.

In some embodiments, audio data, such as lung and heart sound audio, either in raw or processed form, may be audibly conveyed to a user. The audio playback may be performed independently and/or in conjunction with visual display of the data, such as visual representations of the audio and/or motion data, as discussed above.

In some embodiments, concurrently with and/or independently of the lung sound, heart sound, and/or motion data representations, other input data and/or metadata, as described herein, may be displayed or communicated in various formats to aid in providing verification of the AI-based cleaning, annotation, labelling, interpretation, and validation of the data. For example, in some embodiments, additional data, such as additional input data and/or metadata, may be visually overlaid with displayed input data to assist a clinician in reviewing the AI-marked data. In some embodiments, the display or communication of other input data and metadata may include a visual overlay of the other input data over (e.g., on top of) the data marked by the AI system 104b to aid in the process of verifying the cleaning, annotation, labelling, interpretation, and validation of the data. The overlaying of multiple sources of data may or may not be synchronous with the data being marked. In other embodiments, communication of the other input data may include providing one or more additional inputs to a trained machine learning model configured to receive and apply the other input data at one or more hidden layers.

In some embodiments, the disclosed AI systems 104b and/or the disclosed user-interfaces 200-200g may be configured to allow for AI-assisted or augmented cleaning, marking, and interpretation of data. For example, in some embodiments, the user-interface 200-200g may be configured to allow a user to identify a portion of the data and provide that portion of the data to an AI system 104b configured to clean, mark, and/or interpret the identified portion of the data 102. In other embodiments, the AI system 104b is configured to perform one or more automated processes to clean, mark, and/or interpret data 102 and the user-interface 200-200g is configured to provide a user with tools to verify, review, and/or otherwise interact with the automated classifications generated by the AI system 104b.

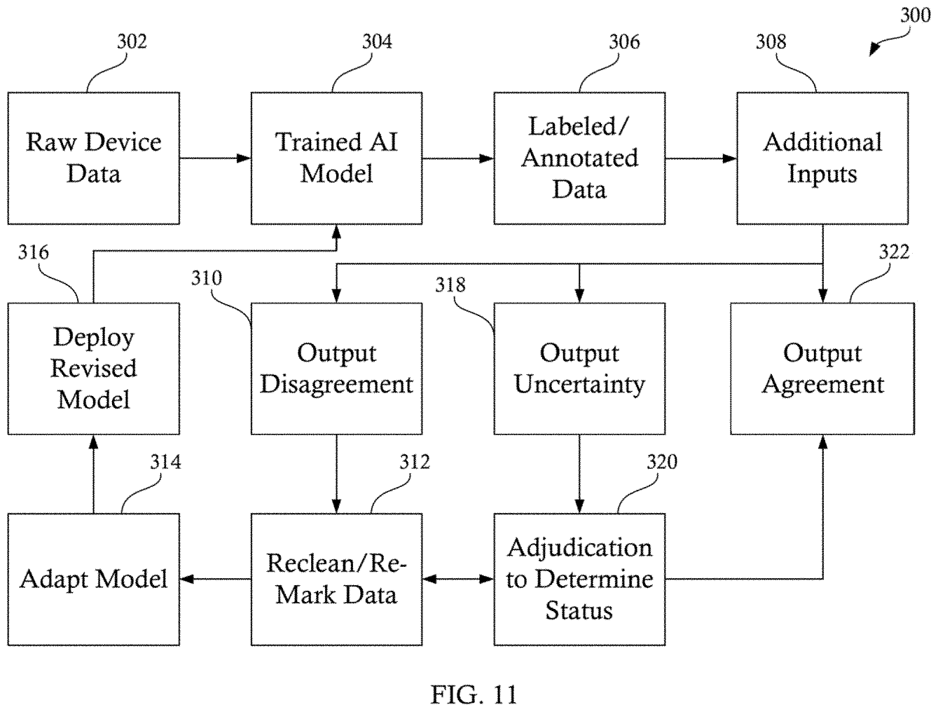

FIG. 11 is a process flow 300 illustrating a computer-implemented machine learning method of generating cleaned and marked data for use in additional machine learning tasks, in accordance with some embodiments. The process flow 300 is similar to the process flows 100, 100a discussed above in conjunction with FIGS. 1-4, and similar description is not repeated herein. The received raw data 302, such as physiological data obtained from one or more devices, is provided to a trained IA model 304 configured to provide cleaning, marking, and/or interpretation of the raw data.

In some embodiments, the AI system 304 includes one or more adaptive properties configured to aid in efficient integration of inputs in the data cleaning and marking process(es). The adaptive properties may include, but are not limited to, transfer learning, adaptive modeling, preference prediction, etc. Transfer learning utilizes a trained machine learning model for one dataset to process another dataset, at the cleaning and marking process. For example, newly acquired datasets may be marked by one or more models) trained on previous datasets input data. Adaptive modeling applies learning feedback to a trained model when the trained model outputs are revised or overridden. Adaptive modeling may be implemented to improve the machine learning algorithms. For example, as the data marking process iterates with new data, disagreements between models (and/or other sources) are identified and subsequently adjudicated, and learning feedback is subsequently applied to the trained models.

For example, as illustrated in FIG. 11, after generating labelled and/or annotated data 306, additional inputs 308 may be received by a training system configured to generate and/or refine the trained AI model 304. The additional inputs 308 may include, for example, cleaning, marking, and/or interpretation of the same or similar datasets as raw device data 302. The additional inputs 308 may be compared to the labelled and/or annotated data 306 to determine agreement between the output of the trained AI system 304 and the additional inputs 308. A comparison between the AI generated data 306 and the additional inputs 308 may identify output disagreement 310, output uncertainty 318, and/or output agreement 322.

In embodiments including output disagreement 310, one or more portions of the additional inputs 308 clean, mark, or interpret the raw data 302 differently than the trained AI model 304. In embodiments including additional inputs 308 having a high confidence value, the data set 302 may be re-cleaned and/or re-marked based on the additional inputs 308 (e.g., assigning the values in the additional inputs 308 to the data set 302, performing cleaning, marking, or interpretation using a different trained AI model, etc.). The re-cleaned and/or re-marked data is used to adapt 314 the trained AI model 304, for example, by providing a set of training data including the re-cleaned and/or re-marked data to a training system. The revised AI model is deployed 316 and replaces the existing trained AI model 304. The revised AI model is applied to future sets of received data.

In embodiments including output uncertainty 318, one or more adjudication processes 320 may be applied to reconcile the disagreement between the trained AI model 304 and the additional inputs 308. For example, if the additional inputs 308 include a confidence threshold equal to or below the confidence threshold of the trained AI model 304 for the AI-generated data 306, one or more adjudication processes 320 may be applied to select the correct cleaning and/or marking. The adjudication processes may be automated processes configured to apply trained AI models, traditional algorithms, and/or other data processes and/or may be manual adjudication processes. Once the adjudication process 320 is completed, the AI-generated data may be re-cleaned and/or re-marked 312 as necessary and provided for adaptation 314 of the trained AI model 304, as discussed above.

In some embodiments, an augmented AI system 304 is configured to log specific tools or processes used to analyze input data, such as data 302. For example, in some embodiments, a specific frequency filter may be used for marking, cleaning, or interpreting wheezes, while echocardiograms may be used for cleaning and marking of heart sounds. In some embodiments, the augmented AI system is configured to automatically and/or preferentially pre-processes and/or displays additional information that is historically helpful for clinical interpretation of the AI generated data 306, increasing efficiency of the review and/or re-marking of the data by eliminating the manipulation required to access desired information or use a desired signal processing tool.

In some embodiments, the disclosed interface(s) may be used for processes other than preparing physiological data for machine learning applications. For example, the disclosed processes and system of methods described above may be used in other use cases in addition to cleaning and marking input data to prepare the data for machine learning applications. Other use cases include clinical research, patient care, or other use cases requiring analysis of input data.

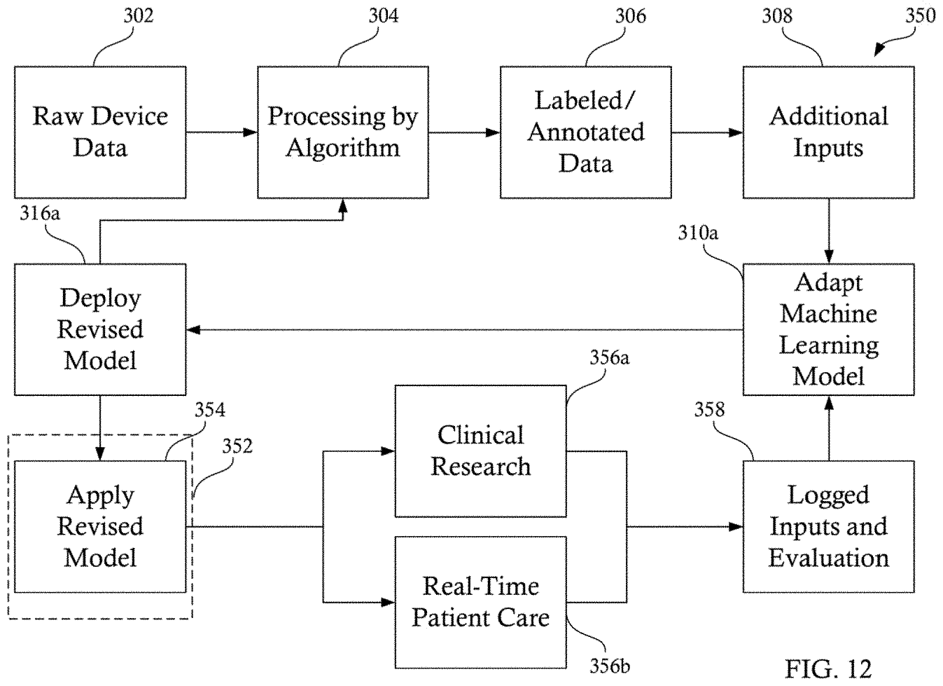

FIG. 12 is a process flow 350 illustrating a method of generating one or more additional machine-learning algorithms using machine cleaned and marked data, in accordance with some embodiments. The process flow 350 is similar to the process flow 300 discussed in conjunction with FIG. 11, and similar description is not repeated herein. The additional inputs 308a may be provided to conform input data and/or trained AI systems 304a, 356a, 356b to accommodate a specific use case and/or specific parameters. As illustrated in FIG. 12, adapted and/or revised AI models 316a may be deployed 354 to one or more user environments 352. In various embodiments, the deployed 354 models may include clinical research models 356a, real-time patient care models 356b, and/or any other suitable models. In some embodiments, clinical research models 356a may be configured to receive historical data from sample populations for clinical review, prediction, etc. and/or may be configured to coincide with experimental applications of data and/or models. In some embodiments, real-time patient care models 356b are configured to apply proven AI systems for data cleaning, marking, interpretation, clinical diagnosis, assisted diagnosis, predictive diagnosis, care recommendations, and/or any other suitable real-time patient care application.

In some embodiments, actions and/or preferences applied during processing are recorded 358 and are used to train additional AI systems to improve the prediction of preferences. For example, in some embodiments, a user of an augmented AI system may mark heart sound data visually on a spectrogram while simultaneously displaying an echocardiogram synchronized with the heart sounds. Concurrently, appropriate signal processing filters may be provided on the user interface to better accentuate the heart sounds of interest and the corresponding portion of the echocardiogram of interest. The selection of the signal processing filters and/or the marking of the heart sound by the user may be recorded and logged for use as training data for training one or more AI systems, for example, an AI system to predictively mark heart sound data configured to the user's preferences and/or to pre-apply the appropriate signal processing filters to accentuate the heart sounds of interest and the corresponding portion of the echocardiogram of interest.

In some embodiments, the augmented AI system may record the time spent by a user on a specific type of data while on the user interface. The augmented AI system may also record the specific manipulation of the data performed using the user interface. The augmented AI system may include one or more trained models configured to utilize this information to apply the correct physician billing code, for example, which may be based on time spent and/or type of work (“evaluation and management”) performed. The augmented and adaptive AI system can adapt to the users' preferences and work habit to make medical coding and billing faster and more accurate.

In some embodiments, methods and processes are applied to maintain subject privacy, data security, and data integrity. The augmented AI system is configured to maintain subject privacy, data security, and data integrity. Subject privacy and data security are maintained by preventing unauthorized access to personally identifiable information using encryption technologies and implementing policies. Optionally, machine learning algorithms may be used to eliminate and/or hide physiological signals that may render a subject identifiable. These physiological signals include but are not limited to speech.

Data integrity may optionally be provided by blockchain technology that optionally includes node-based algorithmic data validation to verify input data modifications, changes in cleaning, marking, and validation of the input data, and the source of inputs.

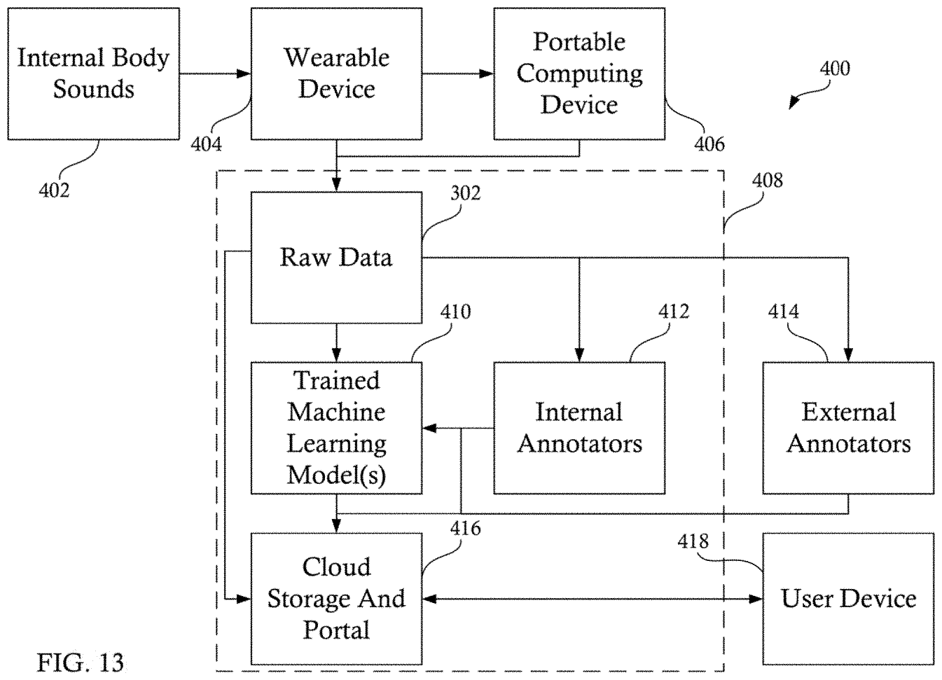

FIG. 13 illustrates one embodiment of a system configured with multiple stages of processed data stored in memory as datasets, in accordance with some embodiments. Physiological data 302 representative of internal body sounds 402 may be collected by a wearable device 404 and transmitted to an AI-enabled environment 408. The raw data 302 may be transmitted directly from the wearable device 404 to the AI-enabled environment 408 and/or may first be provided to a portable computing device 406 that is configured to transmit the raw data to a separate AI-enabled environment 408. Although embodiments are illustrated herein with a separate AI-enabled environment 408, it will be appreciated that the AI-enabled environment 408 may be separate from and/or formed integrally with the wearable device 404 and/or the portable computing device 406.

In some embodiments, the raw data 302 may be provided to various levels of processing within the AI-enabled environment 408. For example, in the illustrated embodiments, a trained AI model 410, one or more internal annotators 412, and/or one or more external annotators (located outside of the AI-enabled environment 408) are configured to clean, mark, interpret, and/or otherwise interact with the raw data 302. The trained AI model 410 may be similar to the trained AI models previously discussed herein and the internal annotators 412 and/or external annotators may utilize augmented AI systems for marking and/or annotation of the raw data 302.

For example, in some embodiments, data processing may occur at an input of each stage 410-414 to clean and/or mark data, as discussed above, in a manner and level associated with the utility of the stored dataset. In some embodiments, additional processing can occur within the output of each stage 410-414 in preparation for the requirements of the following stage, I/O system, or algorithm input. Different levels of permissions can be assigned to users for access to the different stored datasets since each stage 410-414 may have different risk levels associated with privacy. Users may be annotators, researchers, or clinicians, may be internal and/or external employees of a company or other entity, may have different levels of credentials (such as completed privacy training as a requirement for access to the different stored datasets), etc. Access to different stages may have different logging requirements to track access. Trained AI systems (e.g., trained machine learning algorithms) may use data from one or more of the stages for training and/or processing.

In some embodiments, the raw data 302 may contain protected health, security, or private information within the data such as, but not limited to, speech. In some embodiments, this data will only be accessible by properly screened personnel such as personnel with privacy training having sufficient permissions and logging mechanism in place to ensure adequate security. The raw dataset 302 may be provided to with trained AI systems 410 as a source of raw unprocessed data. Further processing of this data may be achieved which may be application specific and stored in other staged processing datasets while maintaining the original raw dataset 302. In the case where data is processed and stored in other datasets, the original raw data 302 is available for future applications and analysis for other applications. For example, data originally collected for a cough study could be re-annotated and/or re-labeled for artificial intelligence training to detect wheezes. Data from this original dataset may be re-accessed many additional times for evaluation of other characteristics of the data and then stored in other datasets for analysis.

In some embodiments, an internal annotation dataset 412 is generated by processing the raw dataset 302 by a trained AI system to clean and/or mark the dataset to remove artifacts that may affect the quality of the data without removing all security and privacy risks. Trained AI systems, such as those discussed above in conjunction with FIGS. 1-4, may be configured to clean the raw data 302 and may further be configured to remove certain features of the raw data 302. Different levels of cleaning may be implemented to satisfy the trade-off between security, privacy, and quality of data. In some embodiments, the cleaning of the raw data 302 is targeted at removing distracting artifacts such as background noise with no requirement of removing security and privacy information since this dataset is protected by adequate security such as controlled access by credentialed internal employees and logging. Cleaning of the raw data 302 may include, but is not limited to, gain, adaptive gain, lowpass filtering, notch filtering, and noise gating.

In some embodiments, an external annotation dataset 414 is generated by processing the raw dataset 302 a trained AI system to clean and/or mark the dataset to remove artifact and privacy information. The external annotation dataset 414 may be de-identified by a trained AI model or other algorithm and used outside of a controlled environment. Additional quality assurance steps may be applied to the external annotation dataset 414 prior to release to an uncontrolled environment. For example, in some embodiments, speech can be detected and flagged. Sections of audio containing speech may be removed, processed with more aggressive filters, and/or processed with trained AI systems specific to the sound of interest. Cleaning of the raw data 302 may include, but is not limited to, gain, adaptive gain, lowpass filtering, notch filtering, and noise gating.

In some embodiments, a cloud storage dataset 416 includes storage of data from multiple datasets which may have additional processing with specific data features extracted in preparation for user consumption. Additional analysis may be performed to extract summary, index, or descriptive data such as heart rate, respiratory rate, respiratory dynamics, I/E ratio, etc. In some embodiments, cloud storage dataset 416 includes data configured to be output to different types of outputs such as headphones, displays, etc. Different means of processing (e.g., different trained AI systems) may be applied to the data depending on the security level of risk for the given output modality. For example, in some embodiments, an output may include the display of a spectrogram on a user device 418, where the risk is low that speech can be discernible and output of raw audio where the risk is high that speech is discernable. The raw audio output may be preprocessed or extracted from a stage using trained AI systems that aggressively make speech indiscernible, while the data applicable to the spectrogram may be preprocessed or extracted from a stage with less aggressive or no mitigation. Any number or types of outputs are anticipated. In various embodiments, speech mitigation may include techniques such as standard filtering, adaptive filtering, spectral gating, noise gating, speech detection, and trained AI models that render speech indiscernible. These techniques may include standard and/or adaptive algorithms and models and may be configured to affect the whole data file and/or process selective parts of the data.

In some embodiments, additional data, which may be collected from other input sources, such as a mobile phone, is stored and then linked to the raw data and/or other collected data such as sensor data from other sources. The additionally collected information may be associated with activities, breathing exercises, diaries, etc. The data may be linked within a dataset. For example, in some embodiments, the dataset may include a temporal such as a time stamp.

In some embodiments, processing can include storing data in smaller units to decrease the amount of speech content to mitigate risk of privacy breaches. In one embodiment, a long data file having a length above a predetermined amount, for example, a data file having a length of 1 minute, can be segmented into separate data files each having a shorter length, such as, for example, 6 files each having a length of 10 seconds. Annotators and labelers may be provided a randomized order of files, causing conversations occurring over multiple files to lose context.

In some embodiments, conditions and/or criteria may be specified in different stages of data processing, such that specific types of data and the accompanying metadata that are desired for a specific application may be extracted for further processing. Exemplary conditions include but are not limited to (1) extract lung sounds only, (2) extract wheezes only, (3) extract only lung sounds with deep breathing (input data associated with specific type(s) of metadata), (4) extract only lung sounds with concurrent heart sounds, (5) extract lung sounds with a spectral power frequency above a certain pre-specified threshold only. As described above, input modifiers may also be used as conditions/criteria based on which input data are directed to the appropriate pathway during staged processing. This staged processing approach according to pre-specified conditions/criteria renders the data processing more efficient by eliminating unwanted data from subsequent processing depending on the staged processing pathway selected.

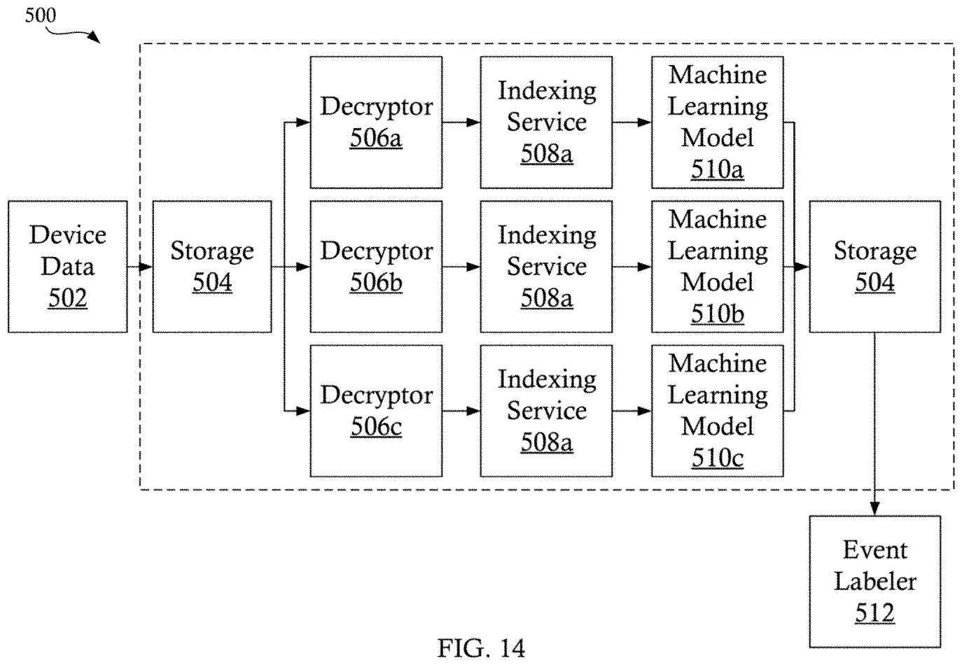

FIG. 14 illustrates a scalable AI-enabled environment 500 configured to provide scalable cleaning, marking, interpreting, and/or other processing of device data 502. Device data 502 may be provided to one or more storage mechanism 504 located within an AI-enabled environment 500. The storage mechanism may include any suitable storage system, such as, for example, one or more cloud repositories, cloud drives, etc. The device data 502 may be stored in raw and/or encrypted form.

In some embodiments, the scalable AI-enabled environment 500 includes a plurality of deployable processing pathways 505a-505c each including various components for preparing and/or processing device data 502 stored in the storage mechanism 504. For example, in the illustrated embodiment, each of the plurality of deployable processing pathways 505a-505c includes a decryptor 506a-506c configured to decrypt encrypted device data 502, an indexing service 508a-508c, and/or a trained AI model 510a-510c. Each of the trained AI models 510a-510c are similar to trained AI models previously discussed, and similar description is not repeated herein. Although embodiments are illustrated herein with three processing pathways 505a-505c, it will be appreciated that processing pathways may be added and/or removed based on the load demands of the AI-enabled environment 500.

In some embodiments, each of the trained AI models 510a-510c is configured to clean, mark, interpret, and/or otherwise process a portion of the device data 502 stored in the storage 504. After being cleaned, marked, interpreted, and/or otherwise processed, the processed data (e.g., outputs of each of the machine learning models 510a-510c) may be stored in a storage mechanism, such as the storage mechanism 504 and/or a different storage mechanism. The stored processed data may be provided to one or more event labelers 512 located outside of the AI-enabled environment 500. In various embodiments, the event labeler 112 may include one or more trained AI models.

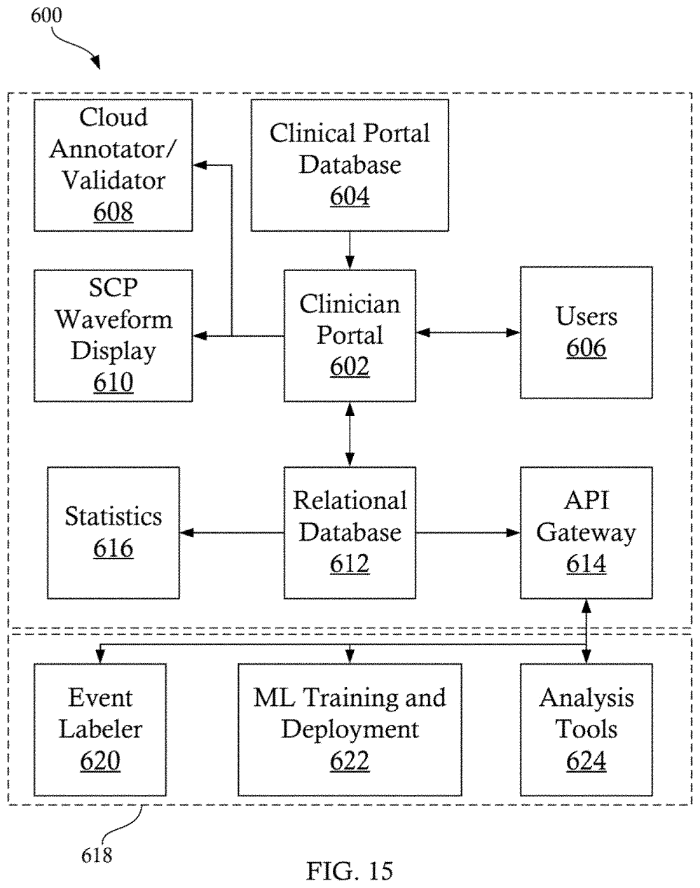

FIG. 15 illustrates an AI-enabled cloud environment 600 for cleaning and validating of device data, in accordance with some embodiments. In some embodiments, the AI-enabled cloud environment 600 includes a clinician portal 602 configured to provide access to one or more users 604. Data corresponding to events that have been previously marked may be distributed by the clinician portal 602 to one or more cloud annotators/validators 608 and/or one or more mechanisms for displaying or presenting the events 610, such as an audio waveform display. The event data may be maintained by a clinician portal database 606.

In some embodiments, the clinician portal is configured to receive updated event data from a relational database 612. The relational database may be any suitable relational database, such as, for example, a Wavpool relational database. The relational database 612 may be in signal communication with a statistics module 616 configured to generate aggregated data statistics and/or an API gateway configured to provide an interface to one or more externally managed systems 618.

In some embodiments, the externally managed systems 618 include an event labeler 620 configured to generate event labels for device data, as discussed in greater detail herein. The event labeler 620 may be configured to provide labeled events to the portal 602 via the API gateway 614 for inclusion in the clinical portal database 606. The API gateway 614 may be configured to provide device data, such as event data, audio data, and/or motion data, to the externally managed systems 618, such as the event labeler 620. The externally managed systems 618 may further include machine learning (or AI) training and deployment 620 of trained AI systems and models and/or application of analysis tools 622, such as ad-hoc analysis tools.

In various embodiments, communications between the externally managed systems 618 and the API gateway 614 may be facilitated by one or more mechanisms, such as, for example, a predetermined library, such as a python library. One or more libraries may be configured to facilitate complex data requests with the AI-enabled cloud environment 600.

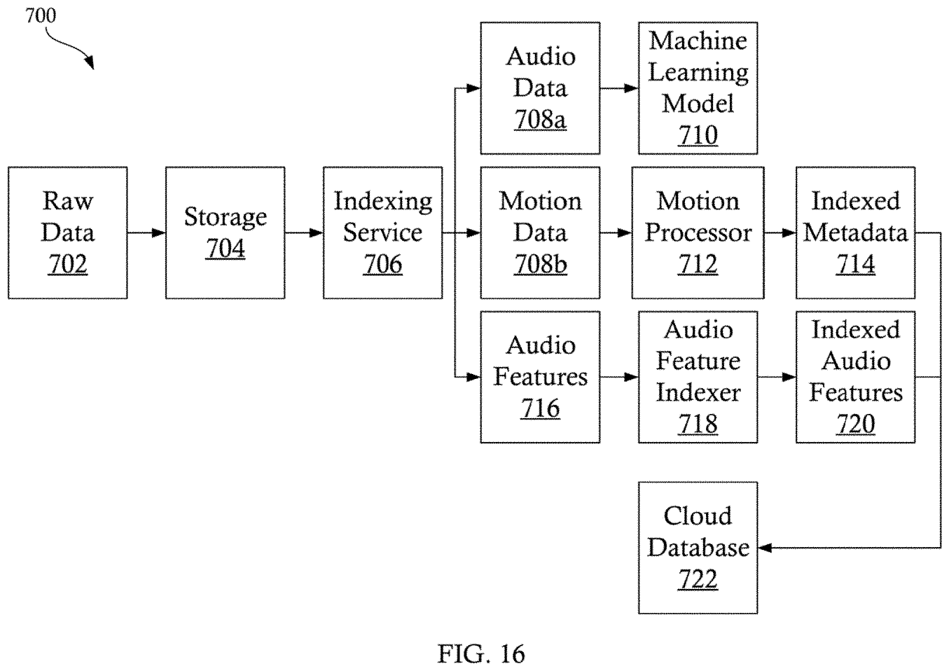

FIG. 16 illustrates a process flow 700 for processing and storage of device data 702, in accordance with some embodiments. Device data 702, such as audio data, motion data, audio features, etc., may be received and stored in a storage mechanism 704. The stored device data 702 may be provided from the storage mechanism 704 to an indexing service 706 configured to provide indexing of the data types included in the device data 702. For example, in some embodiments, the indexing service 706 is configured to identify the audio data, motion data, and audio features included within the data set 702.

Each of the data types within the data set 702 are provided to separate processing pathways for processing. For example, audio data 708a may be provided to a trained AI model 710 configured to clean, mark, interpret, and/or otherwise process the device data 702. The processed data may be provided to the storage mechanism 704 for further processing by additional processing pathways, such as, for example, the audio features processing pathway and/or stored for use in future AI training and deployment.

As another example, in some embodiments, motion data 708b may be processed by a motion processor 712. The motion processor 712 may include a trained AI model configured to clean, mark, and/or interpret motion data included within the device data 702 and/or may include traditional motion processing algorithms. In some embodiments, the motion data 708b is processed and associated with indexed metadata 714 that corresponds to the motion data 708b. The processed motion data 708b and/or the index metadata 714 may be provided to a cloud database 722 for storage. As yet another example, in some embodiments, audio features 716 are provided to an audio feature indexer 718 configured to generate indexed (e.g., timestamped, frequency stamped, etc.) audio features 720. The indexed audio features may be similarly stored in a cloud database 722.

In some embodiments, each of the processing pathways are configured to automatically clean, mark, and/or interpret various types of data to identify events, such as respiratory events (e.g., coughs, wheezes, etc.) included within the data. The output of the trained AI model 410 and/or generated metadata may be used to recursively train AI models for further deployment.

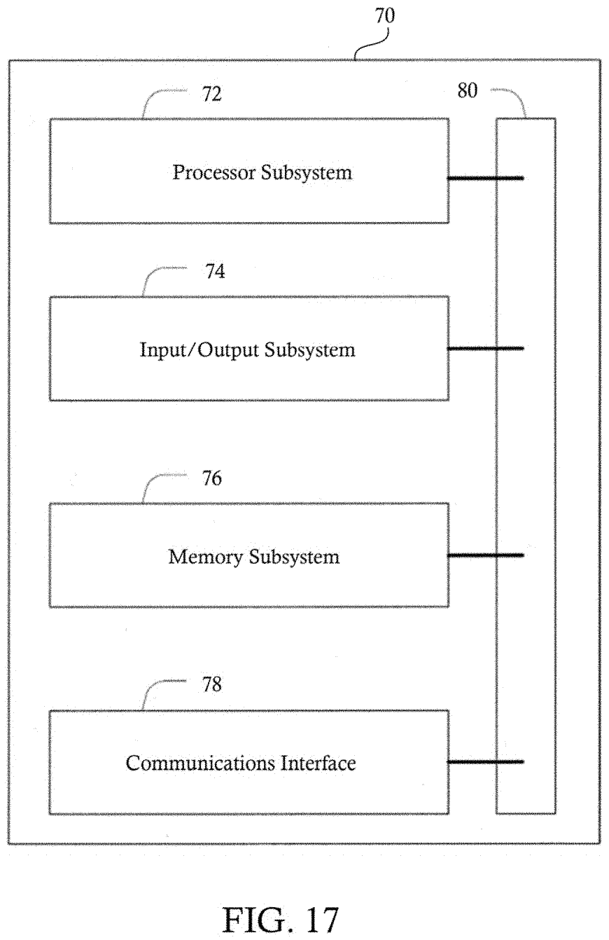

FIG. 17 illustrates a computer system configured to implement one or more processes, in accordance with some embodiments. The system 70 is a representative device and may comprise a processor subsystem 72, an input/output subsystem 74, a memory subsystem 76, a communications interface 78, and a system bus 80. In some embodiments, one or more than one of the system 70 components may be combined or omitted such as, for example, not including an input/output subsystem 74. In some embodiments, the system 70 may comprise other components not combined or comprised in those shown in FIG. 17. For example, the system 70 may also include, for example, a power subsystem. In other embodiments, the system 70 may include several instances of the components shown in FIG. 17. For example, the system 70 may include multiple memory subsystems 76. For the sake of conciseness and clarity, and not limitation, one of each of the components is shown in FIG. 17.

The processor subsystem 72 may include any processing circuitry operative to control the operations and performance of the system 70. In various aspects, the processor subsystem 72 may be implemented as a general purpose processor, a chip multiprocessor (CMP), a dedicated processor, an embedded processor, a digital signal processor (DSP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The processor subsystem 4 also may be implemented by a controller, a microcontroller, an application specific integrated circuit (ASIC), a field programmable gate array (FPGA), a programmable logic device (PLD), and so forth.

In various aspects, the processor subsystem 72 may be arranged to run an operating system (OS) and various applications. Examples of an OS comprise, for example, operating systems generally known under the trade name of Apple OS, Microsoft Windows OS, Android OS, Linux OS, and any other proprietary or open source OS. Examples of applications comprise, for example, network applications, local applications, data input/output applications, user interaction applications, etc.

In some embodiments, the system 70 may comprise a system bus 80 that couples various system components including the processing subsystem 72, the input/output subsystem 74, and the memory subsystem 76. The system bus 80 can be any of several types of bus structure(s) including a memory bus or memory controller, a peripheral bus or external bus, and/or a local bus using any variety of available bus architectures including, but not limited to, 9-bit bus, Industrial Standard Architecture (ISA), Micro-Channel Architecture (MSA), Extended ISA (EISA), Intelligent Drive Electronics (IDE), VESA Local Bus (VLB), Peripheral Component Interconnect Card International Association Bus (PCMCIA), Small Computers Interface (SCSI) or other proprietary bus, or any custom bus suitable for computing device applications.

In some embodiments, the input/output subsystem 74 may include any suitable mechanism or component to enable a user to provide input to system 70 and the system 70 to provide output to the user. For example, the input/output subsystem 74 may include any suitable input mechanism, including but not limited to, a button, keypad, keyboard, click wheel, touch screen, motion sensor, microphone, camera, etc.

In some embodiments, the input/output subsystem 74 may include a visual peripheral output device for providing a display visible to the user. For example, the visual peripheral output device may include a screen such as, for example, a Liquid Crystal Display (LCD) screen. As another example, the visual peripheral output device may include a movable display or projecting system for providing a display of content on a surface remote from the system 70. In some embodiments, the visual peripheral output device can include a coder/decoder, also known as Codecs, to convert digital media data into analog signals. For example, the visual peripheral output device may include video Codecs, audio Codecs, or any other suitable type of Codec.

The visual peripheral output device may include display drivers, circuitry for driving display drivers, or both. The visual peripheral output device may be operative to display content under the direction of the processor subsystem 74. For example, the visual peripheral output device may be able to play media playback information, application screens for application implemented on the system 70, information regarding ongoing communications operations, information regarding incoming communications requests, or device operation screens, to name only a few.

In some embodiments, the communications interface 78 may include any suitable hardware, software, or combination of hardware and software that is capable of coupling the system 70 to one or more networks and/or additional devices. The communications interface 78 may be arranged to operate with any suitable technique for controlling information signals using a desired set of communications protocols, services or operating procedures. The communications interface 78 may comprise the appropriate physical connectors to connect with a corresponding communications medium, whether wired or wireless.

Vehicles of communication comprise a network. In various aspects, the network may comprise local area networks (LAN) as well as wide area networks (WAN) including without limitation Internet, wired channels, wireless channels, communication devices including telephones, computers, wire, radio, optical or other electromagnetic channels, and combinations thereof, including other devices and/or components capable of/associated with communicating data. For example, the communication environments comprise in-body communications, various devices, and various modes of communications such as wireless communications, wired communications, and combinations of the same.

Wireless communication modes comprise any mode of communication between points (e.g., nodes) that utilize, at least in part, wireless technology including various protocols and combinations of protocols associated with wireless transmission, data, and devices. The points comprise, for example, wireless devices such as wireless headsets, audio and multimedia devices and equipment, such as audio players and multimedia players, telephones, including mobile telephones and cordless telephones, and computers and computer-related devices and components, such as printers, network-connected machinery, and/or any other suitable device or third-party device.

Wired communication modes comprise any mode of communication between points that utilize wired technology including various protocols and combinations of protocols associated with wired transmission, data, and devices. The points comprise, for example, devices such as audio and multimedia devices and equipment, such as audio players and multimedia players, telephones, including mobile telephones and cordless telephones, and computers and computer-related devices and components, such as printers, network-connected machinery, and/or any other suitable device or third-party device. In various implementations, the wired communication modules may communicate in accordance with a number of wired protocols. Examples of wired protocols may comprise Universal Serial Bus (USB) communication, RS-232, RS-422, RS-423, RS-485 serial protocols, FireWire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, to name only a few examples.

Accordingly, in various aspects, the communications interface 10 may comprise one or more interfaces such as, for example, a wireless communications interface, a wired communications interface, a network interface, a transmit interface, a receive interface, a media interface, a system interface, a component interface, a switching interface, a chip interface, a controller, and so forth. When implemented by a wireless device or within wireless system, for example, the communications interface 78 may comprise a wireless interface comprising one or more antennas, transmitters, receivers, transceivers, amplifiers, filters, control logic, and so forth.

In various aspects, the communications interface 78 may provide data communications functionality in accordance with a number of protocols. Examples of protocols may comprise various wireless local area network (WLAN) protocols, including the Institute of Electrical and Electronics Engineers (IEEE) 802.xx series of protocols, such as IEEE 802.11a/b/g/n, IEEE 802.16, IEEE 802.20, and so forth. Other examples of wireless protocols may comprise various wireless wide area network (WWAN) protocols, such as GSM cellular radiotelephone system protocols with GPRS, CDMA cellular radiotelephone communication systems with 1×RTT, EDGE systems, EV-DO systems, EV-DV systems, HSDPA systems, and so forth. Further examples of wireless protocols may comprise wireless personal area network (PAN) protocols, such as an Infrared protocol, a protocol from the Bluetooth Special Interest Group (SIG) series of protocols (e.g., Bluetooth Specification versions 5.0, 6, 7, legacy Bluetooth protocols, etc.) as well as one or more Bluetooth Profiles, and so forth. Yet another example of wireless protocols may comprise near-field communication techniques and protocols, such as electro-magnetic induction (EMI) techniques. An example of EMI techniques may comprise passive or active radio-frequency identification (RFID) protocols and devices. Other suitable protocols may comprise Ultra Wide Band (UWB), Digital Office (DO), Digital Home, Trusted Platform Module (TPM), ZigBee, and so forth.

In some embodiments, at least one non-transitory computer-readable storage medium is provided having computer-executable instructions embodied thereon, wherein, when executed by at least one processor, the computer-executable instructions cause the at least one processor to perform embodiments of the methods described herein. This computer-readable storage medium can be embodied in memory subsystem 76.

In some embodiments, the memory subsystem 76 may comprise any machine-readable or computer-readable media capable of storing data, including both volatile/non-volatile memory and removable/non-removable memory. The memory subsystem 76 may comprise at least one non-volatile memory unit. The non-volatile memory unit is capable of storing one or more software programs. The software programs may contain, for example, applications, user data, device data, and/or configuration data, or combinations therefore, to name only a few. The software programs may contain instructions executable by the various components of the system 70.

In various aspects, the memory subsystem 76 may comprise any machine-readable or computer-readable media capable of storing data, including both volatile/non-volatile memory and removable/non-removable memory. For example, memory may comprise read-only memory (ROM), random-access memory (RAM), dynamic RAM (DRAM), Double-Data-Rate DRAM (DDR-RAM), synchronous DRAM (SDRAM), static RAM (SRAM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, disk memory (e.g., floppy disk, hard drive, optical disk, magnetic disk), or card (e.g., magnetic card, optical card), or any other type of media suitable for storing information.

In one embodiment, the memory subsystem 76 may contain an instruction set, in the form of a file for executing various methods, such as methods including implementation of augmented artificial intelligence systems for processing, cleaning, and preparation of data for additional machine learning processing, as described herein. The instruction set may be stored in any acceptable form of machine readable instructions, including source code or various appropriate programming languages. Some examples of programming languages that may be used to store the instruction set comprise, but are not limited to: Java, C, C++, C#, Python, Objective-C, Visual Basic, or .NET programming. In some embodiments a compiler or interpreter is comprised to convert the instruction set into machine executable code for execution by the processing subsystem 72.

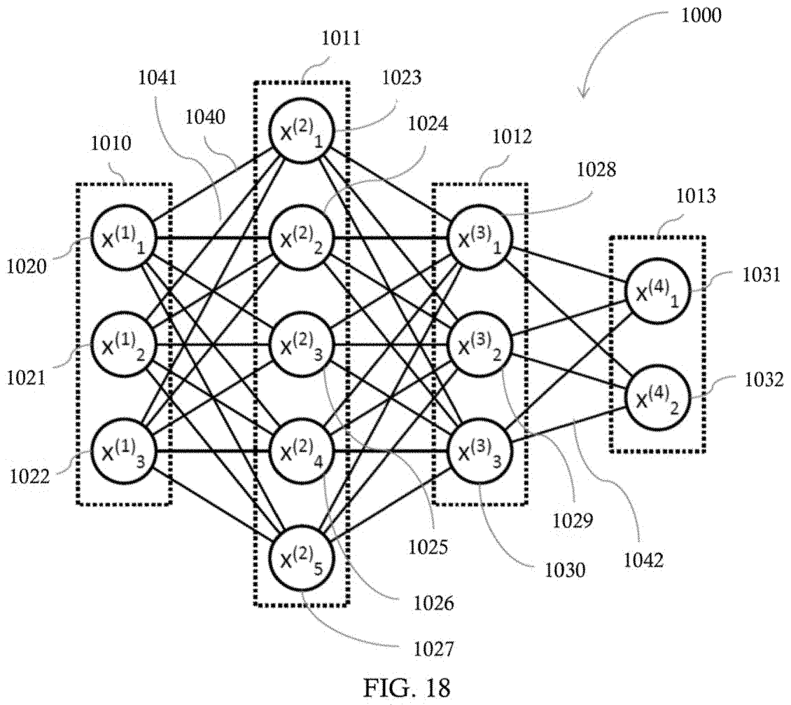

FIG. 18 illustrates an embodiment of an artificial neural network 1000. Alternative terms for “artificial neural network” are “neural network,” “artificial neural net,” “neural net,” or “trained function.” The artificial neural network 1000 comprises nodes 1020-1032 and edges 1040-1042, wherein each edge 1040-1042 is a directed connection from a first node 1020-1032 to a second node 1020-1032. In general, the first node 1020-1032 and the second node 1020-1032 are different nodes 1020-1032, although it is also possible that the first node 1020-1032 and the second node 1020-1032 are identical. For example, in FIG. 18, the edge 1040 is a directed connection from the node 1020 to the node 1023, and the edge 1042 is a directed connection from the node 1030 to the node 1032. An edge 1040-1042 from a first node 1020-1032 to a second node 1020-1032 is also denoted as “ingoing edge” for the second node 1020-1032 and as “outgoing edge” for the first node 1020-1032.

In this embodiment, the nodes 1020-1032 of the artificial neural network 1000 can be arranged in layers 1010-1013, wherein the layers can comprise an intrinsic order introduced by the edges 1040-1042 between the nodes 1020-1032. In particular, edges 1040-1042 can exist only between neighboring layers of nodes. In the displayed embodiment, there is an input layer 1010 comprising only nodes 1020-1022 without an incoming edge, an output layer 1013 comprising only nodes 1031, 1032 without outgoing edges, and hidden layers 1011, 1012 in-between the input layer 1010 and the output layer 1013. In general, the number of hidden layers 1011, 1012 can be chosen arbitrarily. The number of nodes 1020-1022 within the input layer 1010 usually relates to the number of input values of the neural network, and the number of nodes 1031, 1032 within the output layer 1013 usually relates to the number of output values of the neural network.

In particular, a (real) number can be assigned as a value to every node 1020-1032 of the neural network 1000. Here, x⁽ⁿ⁾ᵢ denotes the value of the i-th node 1020-1032 of the n-th layer 1010-1013. The values of the nodes 1020-1022 of the input layer 1010 are equivalent to the input values of the neural network 1000, the values of the nodes 1031, 1032 of the output layer 1013 are equivalent to the output value of the neural network 1000. Furthermore, each edge 1040-1042 can comprise a weight being a real number, in particular, the weight is a real number within the interval [−1, 1] or within the interval [0, 1]. Here, w^{(m,n)}_{i,j} denotes the weight of the edge between the i-th node 1020-1032 of the m-th layer 1010-1013 and the j-th node 1020-1032 of the n-th layer 1010-1013. Furthermore, the abbreviation w⁽ⁿ⁾_{i,j} is defined for the weight w^{(n,n+1)}_{i,j}.

In particular, to calculate the output values of the neural network 1000, the input values are propagated through the neural network. In particular, the values of the nodes 1020-1032 of the (n+1)-th layer 1010-1013 can be calculated based on the values of the nodes 1020-1032 of the n-th layer 1010-1013 by

$${x}_{j}^{( n + 1 )} = f ( {\sum }_{i} {x}_{i}^{( n )} \cdot {w}_{i , j}^{( n )} )$$

Herein, the function f is a transfer function (another term is “activation function”). Known transfer functions are step functions, sigmoid function (e.g. the logistic function, the generalized logistic function, the hyperbolic tangent, the Arctangent function, the error function, the smooth step function) or rectifier functions. The transfer function is mainly used for normalization purposes.

In particular, the values are propagated layer-wise through the neural network, wherein values of the input layer 1010 are given by the input of the neural network 1000, wherein values of the first hidden layer 1011 can be calculated based on the values of the input layer 1010 of the neural network, wherein values of the second hidden layer 1012 can be calculated based in the values of the first hidden layer 1011, etc.

In order to set the values w^{(m,n)}_{i,j} for the edges, the neural network 1000 has to be trained using training data. In particular, training data comprises training input data and training output data (denoted as ti). For a training step, the neural network 1000 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 1000 (backpropagation algorithm). In particular, the weights are changed according to

w′_{i,j}⁽ⁿ⁾=w_{i,j}⁽ⁿ⁾−γ·δⱼ⁽ⁿ⁾·xᵢ⁽ⁿ⁾

wherein γ is a learning rate, and the numbers δ⁽ⁿ⁾ⱼ can be recursively calculated as

δⱼ⁽ⁿ⁾=(Σₖδₖ⁽ⁿ⁺¹⁾·w_{j,k}⁽ⁿ⁺¹⁾)·ƒ′(Σᵢxᵢ⁽ⁿ⁾·w_{i,j}⁽ⁿ⁾)

based on δ⁽ⁿ⁺¹⁾ⱼ, if the (n+1)-th layer is not the output layer, and

δⱼ⁽ⁿ⁾=(xₖ⁽ⁿ⁺¹⁾−tⱼ⁽ⁿ⁺¹⁾)·ƒ′(Σᵢxᵢ⁽ⁿ⁾·w_{i,j}⁽ⁿ⁾)

if the (n+1)-th layer is the output layer 113, wherein ƒ′ is the first derivative of the activation function, and y⁽ⁿ⁺¹⁾ⱼ is the comparison training value for the j-th node of the output layer 1013.

In some embodiments, the neural network 1000 is configured, or trained, to generate an AI model configured to clean, mark, interpret, and/or otherwise process device and/or physiological data. For example, in some embodiments, the neural network 1000 is configured to receive physiological data collected by one or more devices, such as wearable devices, from a first patient. The neural network 1000 can receive the physiological data in any suitable form, such as, for example, raw signal data, filtered data, etc. In various embodiments, the neural network 1000 may be trained to clean, mark, interpret, and/or otherwise interact with device data, as discussed previously herein.

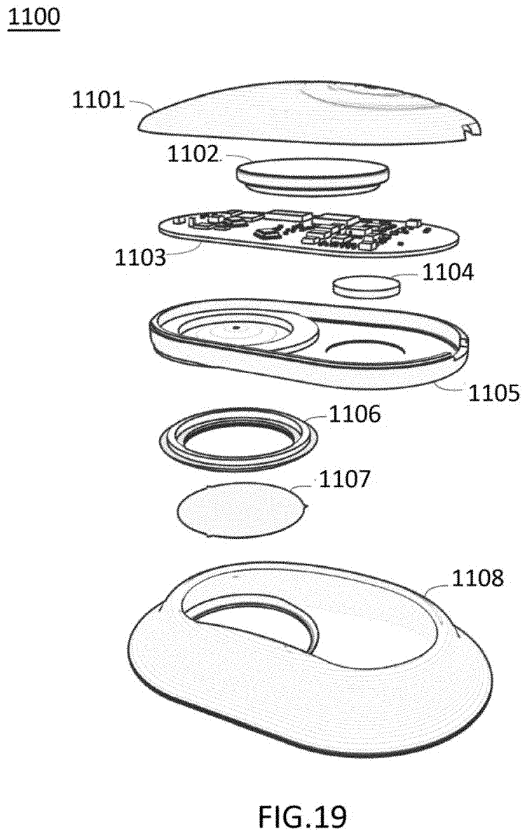

As discussed above, in some embodiments, the AI-enabled systems and methods disclosed herein are configured to utilize physiological data captured by one or more monitoring devices. An exploded view of an exemplary wearable device 1100 is illustrated in FIG. 19. A diaphragm 1107 is configured to be placed in contact with a patient's skin. A diaphragm seal 1106 secures the diaphragm 1107 in place. A chestpiece and bottom housing 1105 is placed above the diaphragm 1107. One or more electronic components 1103 are placed above the chestpiece 1105. A top housing 1101 is placed above the electronic components 1103. A soft enclosure 1108 is placed below the chestpiece and bottom housing 1105. A charging coil 1104 may be in signal communication with one or more of the electronic components 1103.

In some embodiments, the top housing 1101, the bottom housing 1105, and/or the diaphragm 1107 may be formed of a rigid, lightweight polymeric material, although other materials and/or combinations of materials may be used. The soft enclosure 1108 may be formed of a soft silicone or other biocompatible, flexible material. The soft enclosure 108 may be configured to be affixed to a patient's skin using any suitable mechanism, such as an adhesive, straps, clips, etc. The electronic components 1103 are configured to record physiological activities, such as audible sounds, from a patient and generate data that may be used one or more AI-enabled processes, such as diagnosis of a respiratory illness.

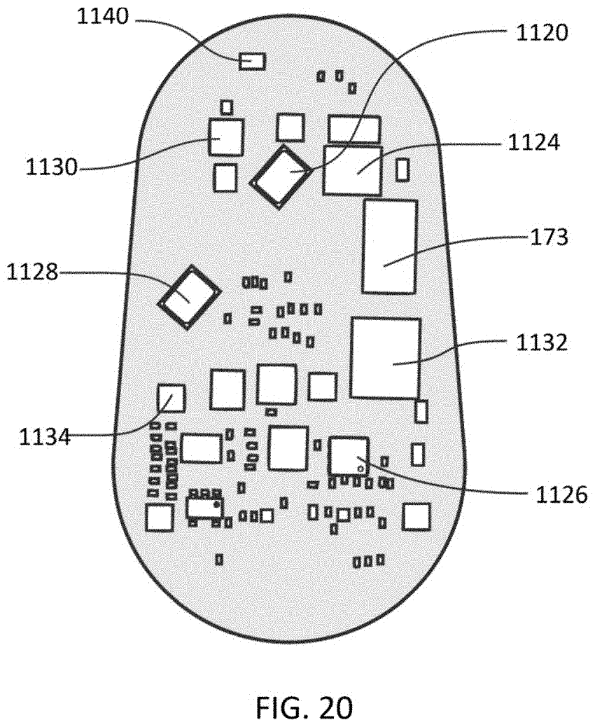

As illustrated in FIG. 20, in various embodiments, the electronic components 1103 include one or more of a chest facing microphone 1120, a background microphone 1122, an RF amplifier 1124, an antenna 1126, a multi-sensor module 1128, a motion sensor, a gyroscope, a magnetometer, an AI-specific processor 1140, and/or any other suitable electronic component. A port hole of the chest facing microphone 1120 may be configured to face the bottom housing 1105 and the port hole of the background microphone 1122 may be configured to face the top housing 1101 when the device 1100 is in a constructed configuration. Although embodiments are discussed herein including a multi-sensor module 1128, it will be appreciated that any suitable number of individual sensors and/or multi-sensor modules may be incorporated into the wearable device 1100.

A battery 1102 is in signal communication with one or more of the electronic components 1103 to power one or more electronic components 1103. The battery 1102 may be any suitable batter, such as a disc battery. A processor 1130 is configured to perform various operations, as described below. Multi-sensor module 1128 includes optional sensors including but not limited to motion sensors, thermometer, and pressure sensors.

In some embodiments, a power management device 1132 is configured to control power levels within electronic components 1103 in order to conserve power. The RF amplifier 1124 and antenna 1126 enable electronic components 1103 to communicate with an external computing device wirelessly (e.g., a smartphone, tablet computer, laptop computer, cloud-based computing system, etc.). Optional USB and programming connectors 1134 enable wired communication with electronic components 1103.

In one embodiment, multi-sensor module 1128 includes a motion sensor module including one or more accelerometers, a gyroscope, and a magnetometer. In one embodiment, a first accelerometer and a gyroscope may be provided on a first chip and a second accelerometer and a magnetometer may be provided on a second chip. By providing the accelerometer and the gyroscope together on a first chip, misalignment of the axes of the sensors is avoided. Similarly, by providing the second accelerometer and the magnetometer together on a second chip, misalignment of the axes of those sensors is avoided. While including multiple sensors on a single chip provides the advantages noted, in other embodiments the sensors are separately affixed to the electronics board. In one embodiment, the elements of the motion sensor module can be set to collect data at a frequency of 2 kHz. In other embodiments, the elements of the motion sensor module 317 collect data at any appropriate frequency, such as 1 kHz, 2 kHz, 3 kHz, 4 kHz, or 5 kHz.

In one embodiment, a motion sensor module may include four sensors, three positioned such that they provide motion data in nine degrees of freedom and a fourth configured to de-noise the concurrent motions. In some embodiments, an accelerometer and a gyroscope are positioned to sense linear and angular motion of a chest wall. Further, a magnetometer may be used to gather data that can be used to characterize non-chest wall motions such as walking, jumping, or ambulating with a walker, based on the linear and angular vectors of the motions. In some embodiments, an additional accelerometer may be used to gather data used to detect heart rate based on concurrent movement of the chest wall. Other applications of multi-axis motion sensing include, but are not limited to, detecting postures and specific motions during physical therapy. By placing additional motion sensors along a different axis than the motion sensors used for chest wall motion measurements, the relative contribution of each type of motion to each vector can be computed, so that multiple motions can be classified.

The data captured by motion sensor module may be used to, for example, determine the amplitude of each breath, the duration of inhalation and exhalation of each breath, and the duration of the interval between breaths, as well as the variability of these parameters. Further, in users wearing more than one wearable device 1100, the respiratory pattern may be further characterized by the movement of different parts of the torso, including the abdominal area and the chest wall. As will be described further herein. This information may be used in combination with the audio data captured by microphones 1120, 1122 to characterize abnormal respiratory sounds and assess the risks associated therewith.

The concurrent motion monitoring may be configured to obtain data for respiratory monitoring. For example, a change in posture, chest wall movement, and ambulatory pattern (which includes but is not limited to gait, activity level, and timing of ambulation), can be monitored for: (1) detection of respiratory decompensation; (2) adjustment of medications, such as pain medications that can reduce respiratory drive; (3) dynamic feedback for physical therapy and pulmonary rehabilitation, etc.

In some embodiments, one or more sensors, such as a multi-sensor module 1128, are configured to perform data acquisition. Physiological signals, such as sound, is received by one or more sensors, for example, one or more microphones (e.g., chest facing microphone 1120 and/or background microphone 1122) that are configured to convert acoustical energy into electrical energy, piezoelectrical elements, etc. The chest facing microphone 1120 and/or the background microphone 1122 may include a capacitor-based microphone, a contact accelerometer, and/or any other suitable audio/vibration capture device. In some embodiments, one or more sensors are configured to obtain motion data, pressure data, temperature data and/or additional physiological and/or environment data. Signals from each of the microphones 1120, 1122 and/or one or more sensors (e.g., multi-sensor module 1128) may be transmitted to one or more additional processing components, such as an A-D converter and/or an electrical bus interface.

In some embodiments, data obtained by the wearable device 1100 may be processed (e.g., cleaned, marked, interpreted, etc.). The processing may be performed by an onboard processor (e.g., processor 1130) or a separate processor located in a local computing device, remote computing device, and/or cloud computing device.

In some embodiments, one or more physical filters may be used to perform signal correction, noise correction, or other signal processing tasks. For example, in various embodiments, a physical filter may include a linear continuous-time filters, a low-pass filter, a high-pass filter, an electronic filter, a digital filter, a mechanical filter, and/or any other suitable filter type and/or mechanism.

The processor 1130 may include one or more additional processing components, such as, for example, a digital signal processor, memory, a wireless module, etc. The processor 1130 may include a programmable processor, such as, for example, a Cypress programmable system-on-chip, field programmable gate array with integrated features, a wireless-enabled microcontroller coupled with a field programmable gate array, etc. The wireless module may use any suitable transmission mechanism, such as, for example, Bluetooth Low Energy, and may include an integrated balun and a fully certified Bluetooth stack.

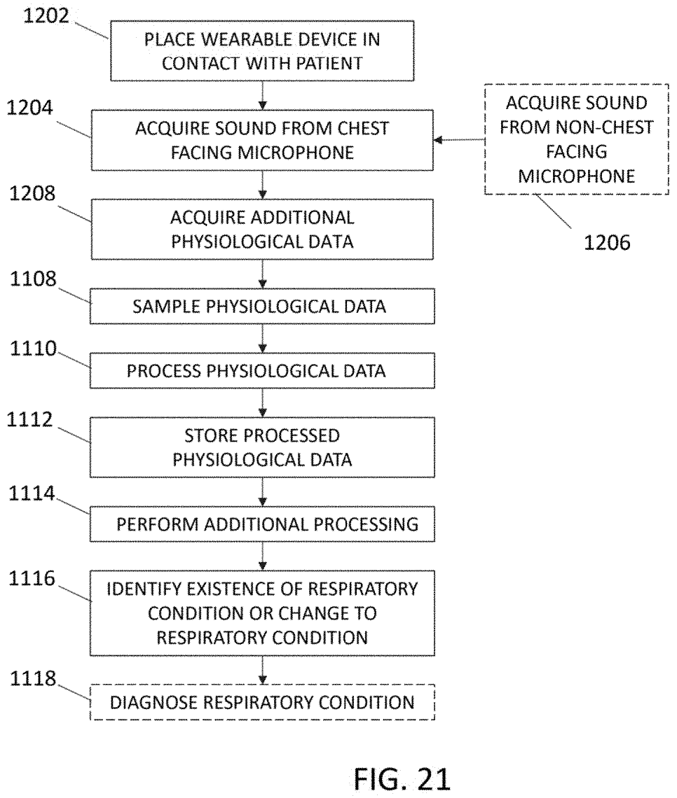

FIG. 21 is a flowchart illustrating a process of collecting and processing physiological data using a wearable device, in accordance with some embodiments. At step 1202, wearable device 1100 is placed in contact with a patient (for example, in direct contact with a patient's skin). Wearable device 1100 may include an adhesive to hold it in contact with the patient, although other forms of adherence may be used. Wearable device 1100 is placed so that chest facing microphone 1120 faces the patient and background microphone 1122 does not face toward the patient.

At step 1204, sound from chest facing microphone 1120 is acquired. At step 1206, sound from background microphone 1122 is acquired. At step 1208, additional physiological data, such as motion data, is acquired by one or more sensors. Received physiological data may be provided to a processor 1130. The processor 1130 is configured to sample the physiological data. The data sampling may occur at single sampling rate, for example at 20 kHz, and/or variable sampling rates based on data sources, types, etc. In some embodiments, data is sampled for a predetermined time period, such as, for example, twenty seconds.

In some embodiments, the processor 1130 is configured to perform cleaning, marking, and/or interpreting of the processed data, for example, as illustrated at step 1210. The cleaning, marking, and/or interpreting may be performed using one or more known processes (such as noise cancelling processes) and/or using an AI-enabled system as previously discussed.

In some embodiments, audio data is processed in order to detect certain sounds associated with breathing (and/or associated with breathing difficulties). Processing at step 1210 may include, for example, Fast Fourier Transform. Processing may also include, for example, digital low pass and/or high pass Butterworth and/or Chebyshev filters. Processing may include application of traditional algorithms and/or trained AI models, as discussed above.

At step 1212, data may be stored in memory, such as, for example, on-board memory formed integrally with the wearable device 1100, memory in a local and/or remote computing device, and/or cloud-based memory systems. Although step 1212 is illustrated after step 1210, it will be understood that step 1212 may be performed concurrently and/or prior to step 1210.

In some embodiments, data stored in memory includes “raw” data, i.e., the actual physiological data obtained by the wearable device such as a recording of sounds that have been sampled by a microphone 1120. In some embodiments, the most recent 20 minutes of raw audio data is stored in memory. The data is stored in a first in, first out configuration, i.e. the oldest data is continuously deleted to make room in memory for data that is newly and continuously acquired. The second type of data that is stored in memory is processed data, i.e. data that has been subjected to a form of processing. Examples of this type of processed data includes the examples set forth above. In some embodiments, 20 seconds of processed audio data is stored in memory and may be stored in a first in, first out configuration.

At step 1214, additional processing of the physiological data is performed. For example, the processed data may be evaluated to determine if an “abnormal” respiratory sound has been captured by microphone 1120. Examples of an “abnormal” respiratory sound include a wheeze, a cough, rhonchi, labored breathing, or some other type of respiratory sound that is indicative of a respiratory problem. In some embodiments, a AI-enabled or AI-augment model is configured to generate a spectrogram from cleaned data. The spectrogram may correspond, for example, to the 20 seconds worth of processed data that has been stored in memory. The spectrogram may be evaluated, for example by the same AI-enabled model, using a set of “predefined mathematical features”.

The “predefined mathematical features” are generated from multiple “predefined spectrograms”. Each “predefined spectrogram” is generated by processing data that is known to correspond to an irregular respiratory sound (such as a wheeze). The predefined spectrograms may be generated using trained AI models and/or trained AI-augmented processes, as discussed above. The predefined spectrograms can be patient specific. For example, a trained AI model may be applied to data from particular patient who will wear the wearable device 1100. The predefined spectrograms can also be population based, e.g., based on data from one or more persons other than the individual who will wear the wearable device 1100. In some embodiments, the predefined spectrograms are based on both patient specific and population based data.

A set of mathematical features can be extracted from each predefined spectrogram. Mathematical feature extraction is known to one of ordinary skill in the art and is described in various publications, including 1) Bahoura, M., & Pelletier, C. (2004, September). Respiratory sounds classification using cepstral analysis and Gaussian mixture models. In Engineering in Medicine and Biology Society, 2004. IEMBS′04. 26th Annual International Conference of the IEEE (Vol. 1, pp. 9-12). IEEE; 2) Bahoura, M. (2009). Pattern recognition methods applied to respiratory sounds classification into normal and wheeze classes. Computers in biology and medicine, 39(9), 824-843; 3) Palaniappan, R., & Sundaraj, K. (2013, December). Respiratory sound classification using cepstral features and support vector machine. In Intelligent Computational Systems (RAICS), 2013 IEEE Recent Advances in (pp. 132-136). IEEE; 4) Mayorga, P., Druzgalski, C., Morelos, R. L., Gonzalez, O. H., & Vidales, J. (2010, August). Acoustics based assessment of respiratory diseases using GMM classification. In Engineering in Medicine and Biology Society (EMBC), 2010 Annual International Conference of the IEEE (pp. 6312-6316). IEEE; and 5) Chien, J. C., Wu, H. D., Chong, F. C., & Li, C. I. (2007, August). Wheeze detection using cepstral analysis in gaussian mixture models. In Engineering in Medicine and Biology Society. All of the above references are hereby incorporated by reference in their entireties.

The set of mathematical features are derived from the inherent power and/or frequency of the predefined spectrogram of data clusters using mathematical methods that include but are not limited to the following: data transforms (Fourier, wavelet, discrete cosine) and logarithmic analyses. The set of mathematical features extracted from each predefined spectrogram can vary by the method with which each feature in the set is extracted. These features may include, but are not limited to, frequency, power, pitch, tone, and shape of data waveform. See Lartillot, O., & Toiviainen, P. (2007, September). A Matlab toolbox for musical feature extraction from audio. In International Conference on Digital Audio Effects (pp. 237-244). This reference is hereby incorporated by reference in its entirety.

For example, in one embodiment, a first set of two mathematical features are extracted from a predefined spectrogram using statistical mean and mode. A second set of two mathematical features are extracted from the same predefined spectrogram using statistical mean and entropy. The set of mathematical features can also vary by the number of features in each set of mathematical features. For example, in one embodiment, a set of twenty mathematical features are extracted from a predefined spectrogram. In another example, a set of fifty mathematical features are extracted from the same predefined spectrogram. Additionally, the mathematical features may vary by the segment lengths of the predefined spectrogram with which the mathematical features are extracted. For example, a mathematical feature extracted from one-second segments of the predefined spectrogram using a statistical method is different from a mathematical feature extracted from five-second segments of the predefined spectrogram using the same statistical method.

The set of mathematical methods used to extract the “predefined mathematical features” is the “pre-specified feature extraction”. In one exemplary embodiment, the “pre-specified feature extraction” is developed using mel-frequency cepstral coefficients and is optimized using machine learning methods that include but are not limited to the following: support vector machines, decision trees, gaussian mixed models, recurrent neural network, semi-supervised auto encoder, restricted Boltzmann machines, convolutional neural networks, and hidden Markov chain (see above references). Each machine learning method may be used alone or in combination with other machine learning methods.

The “predefined mathematical features” are derived from multiple predefined spectrograms in the following manner. A feature extraction method, as defined above, is used to extract a set of mathematical features from each predefined spectrogram corresponding to a type of respiratory sound. Multiple features are evaluated in this manner. The features are then plotted together from multiple respiratory sound types in order to perform cluster analysis in the nth dimension (n being the number of features extracted). For example, if three features were extracted for analysis from each data file, each data file would correspond to one point in three-dimensional space, each axis representing the value of a particular feature. Thereafter, one example of algorithm generation attempts to find a hyperplane in this three-dimensional space that maximally separates clusters of points representing specific sound types. For example, if data points from wheeze files cluster in one corner of this three dimensional space while those from cough files cluster in another, a plane that separates these two clusters would correspond to an algorithm that distinguishes the two and is able to classify these sound types into two groups. This analysis can be extrapolated to as many features as needed, n, thereby moving the analysis into nth dimensional space. This allows differentiation of each sound type based on its unique feature set. The algorithm that generates outputs (sets of mathematical features) that are most similar to each other is selected as the “pre-specified algorithm” as described above. For example, ten sets of twenty statistical features is extracted from ten predefined spectrograms corresponding to wheezing using different algorithms. The algorithm that extracts ten sets of features that are the most similar to each other is selected as the “pre-specified algorithm.” In an exemplary graphical representation of classification, lines represent the “pre-defined algorithm” in classifying data in multiple dimensions in accordance with an exemplary embodiment. Next, the “average” of the sets of mathematical features extracted with the “pre-specified algorithm” is selected as the “predefined mathematical features”. Here, “average” is defined by mathematical similarity between the “predefined mathematical features” and each set of mathematical features from which the “predefined mathematical features” derives from.

Evaluation of a spectrogram with a predefined spectrogram may be on several bases. A spectrogram is processed by the “pre-specified feature extraction” method to generate a set of mathematical features. The set of mathematical features is then compared to sets of “predefined mathematical features”, of which each set corresponds to a specific type of sound. If the similarity between the set of mathematical features extracted from a spectrogram and the predefined mathematical features of a type of respiratory sound goes past certain thresholds, then it is determined that the corresponding type of respiratory sound has been emitted. By saying ‘goes past” what may be meant is going above a value. What may alternatively be meant is going below a value. Thus, by portions of the spectrogram going above or below portions of the predefined spectrogram associated with possible abnormal respiratory sounds, it is determined that an abnormal respiratory sound may have occurred.

A variety of factors can be used to identify, from the available predefined spectrograms, those that a particular patient's data should be compared to and to otherwise classify respiratory sounds. For example, when the wearable device is used post-surgery, predefined spectrograms collected from a subject with a similar surgical anatomy can be used. Selecting appropriate comparison spectrograms in this way may provide more accurate results because general population data may be inappropriate for the post-surgery period. In some embodiments, the motion data is also compared to data gathered from patients with similar anatomy and/or suffering from similar conditions.

In addition, the appropriate predefined spectrograms can be selected based on a pulmonary disease experienced by the patient. For example, the predefined spectrograms can be filtered to those that were captured from patients with COPD. Respiratory sounds are often diminished in patients with severe COPD. COPD also affects pulmonary mechanics. The chest wall is expanded at baseline in patients with COPD, which is termed “barrel chest”. This affects angular and linear displacements, and subsequent calculation of tidal volume and airflow rate. The severity of COPD can be determined from past medical records, and for patients without adequate prior medical evaluation, from smoking history. Selecting the predefined spectrograms by matching COPD history or smoking history can help ensure that the most relevant factors are considered.

An exemplary application involves a patient with esophageal surgery, which puts the patient at high risk of chemical pneumonitis from surgical site leaks. With the development of a surgical leak, this exemplary patient's lung sound generates a specific signature. Concurrently, the patient may have increased respiratory rate and decreased tidal volume. However, the patient may have a barrel chest as a result of severe COPD. Therefore, decreased tidal volume will not result in a decrease in chest wall movement that would otherwise be expected from a patient without COPD. As described above, the predefined spectrograms may be derived from a plurality of populations, such that the difference in boundary conditions for patients with and without COPD could be gathered and applied for the exemplary case.

Additionally, physiological data can be used to distinguish edematous chest wall or lungs from a chest wall and lungs that do not have an edema. This information can be used to refine or filter the spectrograms to which the patient's respiratory sounds will be compared. Because an edematous chest wall transmits sound differently than a chest wall without edema, comparison with data collected from subjects with a similar condition can further enhance the accuracy of the determination of abnormal respiratory sounds.

In addition, the predefined spectrograms can be filtered based on the patient's history of heart failure. These patients may experience wheezing due to bronchospasm or decompensated heart failure, which often also leads to an increase in weight. Based on sound alone, wheeze due to bronchospasm is hard to distinguish from a cardiac wheeze. In these patients, classification of respiratory wheezes vs. cardiac wheezes may take into account information available elsewhere in a patient's medical records. One key differentiator is a patient's past medical history. A marker of worsening heart failure is increasing body weight. This information can be used to adjust the threshold of classification. For example, in a patient without a history of heart failure, a wheeze can be classified as a wheeze due to bronchospasm regardless of the amount of weight gain. However, in a patient with heart failure, a significant weight gain (i.e., two bounds or more) will lead to the classification of a wheeze as a cardiac wheeze. Compared to patients without a history of heart failure, in patients at risk of decompensated heart failure, a smaller change in weight will lead to a classification of cardiac wheeze rather than non-cardiac wheeze.

Wheezes and other respiratory sounds can further be classified based on at what point in the respiratory cycle the wheeze occurs (e.g., during the inhalation or expiration phase). In various embodiments, it may be determined in which portion of the cycle the respiratory sound occurs based on additional physiological data.

In some embodiments, patient specific predefined spectrograms are acquired prior to a surgery to provide a pre-surgery benchmark for post-surgery monitoring. In addition to acquiring pre-surgery spectrograms, other pre-surgery information may be gathered. For example, the patient's chest wall movement data, heart rate, respiratory rate, and ambulatory patterns including but not limited to posture and gait. In addition to being used as benchmarks, this data can be used in the selection of appropriate boundary conditions or benchmark spectrograms for the patient. Alternatively, or additionally, the audio and/or motion data can be compared to data captured after surgery, but at an earlier time, from the same patient.

Other exemplary inputs used for selection of benchmark spectrograms or boundary conditions include video imaging inputs. The inputs could be from a camera of a personal mobile device or a “smart” television in the patient's home. Video input is used to determine the placement of the wearable device 1100 on the patient's chest wall. The video may also be used to correlate sound and motion sensor data to the patient's movements, which includes but is not limited to respiration, posture, and gait. Correlation with video inputs may be incorporated into the calibration process but is not required. Video inputs from the individual may be compared against a population-based database and may contribute to selection of the appropriate boundary conditions.

Once an irregular respiratory sound (such as a wheeze) has been identified using the “predefined mathematical features” the previous 20 (for example) minutes of accumulated raw data that has been stored in memory may receive “further processing.” In one exemplary embodiment, the 20 minutes of raw data is transferred from an internal memory unit to an external computer or cloud environment for more robust processing. In another exemplary embodiment, raw data is subjected to further processing in processor 1130 without being transferred to an external computer.

By implementing a “further processing” step, a first algorithm, such as a first trained AI model, is used to possibly identify an irregular respiratory sound and a second algorithm, such as a second trained AI model, (more robust—i.e. that requires more significant processing than the first model) is applied to the raw data to try to make a more accurate determination as to whether an irregular respiratory sound (such as a wheeze) has indeed occurred. In one exemplary embodiment, a first model generates twenty mathematical features and a second model generates fifty mathematical features (e.g., is more robust). In another exemplary embodiment, the mathematical methods used to extract each mathematical feature in the second algorithm require more processing power than the mathematical methods used in a first algorithm. As such, the second algorithm may be more robust.

Thus, this further processing may include determining whether processed data has passed (i.e. above or below) boundary conditions. The boundary conditions may include one or more of any of the inputs and/or characteristics identified above, such as the mathematical features extracted from the predefined spectrograms. In one embodiment, this is accomplished by pre-specified algorithms previously developed using a machine-learning approach using a deep-learning framework, as discussed above. This involves a multi-layer classification scheme. The variables used in the pre-specified algorithms in the external computer include, but are not limited to, the exemplary variables described above.

In addition to using a spectrogram with the second algorithm, other factors may also be used in the analysis. Exemplary factors include: 1) user inputs, including subjective feelings, rescue inhaler use, type and frequency of medication use, and current asthma status; 2) input from sensors (e.g., accelerometers, magnetometers, and gyroscopes) related to a patient's current physiological status, as will be described in more detail below; 3) environmental inputs available from sensors, which include but are not limited to temperature sensors and barometers; and 4) environmental inputs available from an information source such as the internet. In other words, other variables may be integrated into the analysis, in place of or in addition to the variables that form the basis of the analysis of the initial processed data (e.g., the 20 seconds of data, for example, discussed above). These factors can also include the patient's demographics, heart rate, surgical type, activity level, posture, gait, medication use, and results of medical imaging.

In one embodiment, medical imaging can be used to derive body tissue composition and anatomy. This information can then be used to define the boundary conditions to which the patient's respiratory sounds are compared.

In another embodiment, the patient's use of medication is used to further define the spectrograms and boundary conditions to which the patient's respiratory sounds are compared. Many common pain medications, including but not limited to opioids and ketamine, can cause respiratory and neurological depression. Respiratory depression may manifest with decreased tidal volume and respiratory flow rate. The wearable device 1100, via the motion sensor module, can measure body motion and the resulting data may be used to detect these changes. Comparing the data to spectrograms of user's that are using similar medication may allow for more accurate characterization. Neurological depression may manifest with decreased tidal volume and respiratory flow rate. This condition can also manifest with aspiration and upper airway obstruction, which has an effect on lung sounds in addition to chest wall motion. Neurologic depression also leads to less overall patient movement. The wearable device 1100 can measure body motion and lung sounds and the motion and audio data can be used to detect such changes. Further, in such an embodiment, the patient's medication use data can be correlated with sensor data to provide feedback on the safety of pain medication use.

The information gathered by the wearable device 1100 and/or provided by a patient or caregiver (e.g., patient height, patient weight, patient demographics, medications, surgical information) can also be used to refine and adjust the boundary conditions. For example, the comparison mathematical features extracted from the predefined spectrograms may be adjusted up or down based on data derived from physiological data.

When it is determined that the data has crossed above or below the boundary conditions, an alert or warning can be provided. The alert or warning can be issued to the patient and/or to a physician or caregiver. For example, the wearable device 1100 can issue audible, visual, or tactile feedback, such as by beeping, illuminating one or more lights, or vibrating. Alternatively, the wearable device 1100 can be connected to a computing device, such as a smartphone, via wireless module. As a result, an alert can be issued on the computing device. In some embodiments, the computing device issuing the alert is the external computer. The alert can also be sent to a physician or other caregiver such that the caregiver can contact the patient or notify emergency responders.

The alarm threshold (i.e., the amount of deviation from the boundary conditions required to issue the alarm) may vary from patient to patient. For example, if the patient is using the wearable device 1100 after surgery, the alarm threshold may be lower (i.e., more sensitive) because the patient may be at higher risk than the general population. The threshold may further vary based on the type of surgery and potential complications. For example, a patient at risk of chemical pneumonitis may require a lower threshold.

The “raw” data that may be stored provides multiple functions. For example, it provides an extended period of time for respiratory sound classification. The data may be processed into a spectrogram, and then a second algorithm may be used to analyze the spectrogram, in conjunction with other variables mentioned above. As a further example, the raw data may be used to improve the algorithm. For example, should an abnormal lung sound be recognized, it can serve as a control, and the raw data may be used as a dataset to further refine (or “train”) additional AI-based models.

An exemplary spectrogram based on audio data captured in accordance with an exemplary embodiment is illustrated in FIG. 28. The top portion is obtained from a microphone 1120 facing towards the patient. The bottom portion is obtained from a microphone 1122 facing away from the patient.

Additional algorithms (e.g., traditional algorithms or trained models) can be implemented in accordance with goals of the analysis. For example, in one embodiment, multiple sound samples are obtained and classified into different lung sounds. Next, the samples (spectrograms) are input into a pre-specified classification algorithm to generate a set of mathematical features. The difference between the output of this classification algorithm and the pre-defined mathematical features is used to refine the algorithms. The goal is ensure the classification algorithm has the variables needed to filter out unwanted noises during feature extraction.

Next, the classification algorithm can be applied to additional samples containing both an audio spectrogram and additional user data defined as “boundary conditions” above. The machine learning approach in this case need not focus on feature extraction. Rather, this machine learning approach employs predictive statistical analysis. The basic concept remains the same: the difference between the classification algorithm and the pre-defined answer is used to create and adjust the weight of variables.

For example, in some embodiments, a respiratory condition is detected by identifying how many times a certain type of respiratory sound occurs during a time period (“frequency”). If the number of times the sound is identified in a time period goes past a threshold, then a signal is generated to indicate that an adverse respiratory condition has been detected (or that an adverse respiratory condition has gotten better or worse). By saying “goes past a threshold” what is included is meeting the threshold, going above the threshold, or going below the threshold, depending upon what adverse respiratory conditions are desired to be detected. In a further exemplary embodiment, the number of times a certain type of respiratory sound occurs in a first time period is compared with the number of times the certain type of respiratory sound occurs in a second time period (the first and second time periods may or may not be overlapping, the first and second time periods may or may not be equal). For example, the number of respiratory sounds in a first time period may be compared with the number of respiratory sounds in a second time period greater than the first time period. Comparisons may be with regard to frequency, power, location in the time frame being evaluated, and/or other criteria. In one exemplary embodiment, the first time period may be three hours and the second time period may be 18 hours. These time periods are merely exemplary.

In another exemplary embodiment, respiratory issues are identified based on frequency of audio signal (wheeze frequency ˜300-400 Hz) and the number of times an event occurs (frequency of the event itself).

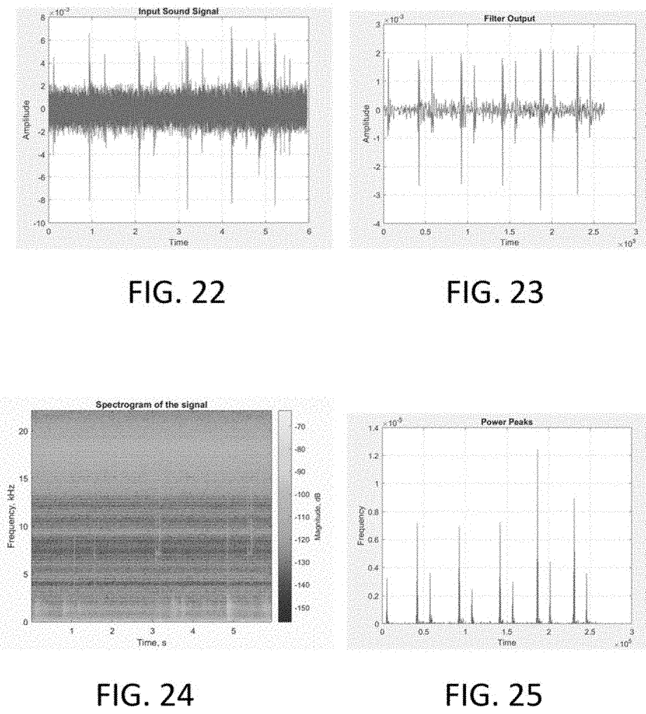

Alternatively, or additionally, the wearable device 1100 can detect and monitor other physiological events. For example, the wearable device 1100 can be used to detect heart rate and heart rate variability of the wearer. As described above, the wearable device 1100 includes two microphones recording two channels of data. The first microphone 1120 is facing the chest wall of the wearer and the second microphone 1122 is facing away from the chest wall and is configured to capture primarily external sounds. FIG. 22 shows an exemplary sample of the two channels overlaid. In order to remove the external noise, the second signal is subtracted from the first signal. Next, a high pass filter is applied to the data, the result is shown in FIG. 23. FIG. 24 shows the same data in the form of a histogram. In the histogram, the high-volume peaks can be clearly seen. Finally, the data is squared to further highlight the heart beats detected by the first microphone 305, as shown in FIG. 25.

After filtering of the data, the peaks can be counted to determine a heart rate. A peak detection algorithm can be used to count the number of peaks at a predefined interval and store this value in a vector. The predefined interval can be any appropriate interval, such as 0.5 seconds. The vector of beats per interval can then be used to identify variability of the heart rate using root mean square of the successive differences method. The vector can also be used to calculate the average beats per minute.

In further embodiments, wearable device 1100 may be configured to detect other heart sounds, such as heart murmurs and changes in the characteristics or rate of heart murmurs over time. The detection of heart sounds (e.g., using audio data from first microphone 1120) along with activity and posture information derived from motion data captured by motion sensor module may aid in the evaluation of diseases, including but not limited to diseases of the heart valve, heart failure, arrhythmias, and cardiac syncope. This may be especially helpful to monitor a patient at home, and to evaluate a patient's response to therapy at home.

In some embodiments, the presence of mouth-breathing can also be detected by comparing the audio data from first microphone 1120 and second microphone 1122. When the differential between lung sounds captured by first microphone 1120 and second microphone 1122 diminishes significantly, mouth breathing may be suspected. This is because the abnormal lung sounds can be transmitted to the ambient environment when the patient's mouth is open, and the sounds can subsequently be captured by the external microphone (e.g., second microphone 1122). Mouth breathing is clinically significant as it may suggest deteriorating respiratory status in a patient. Further, the occurrence of mouth breathing in a patient that is also experiencing adventitious breath sounds in a stationary user (as determined based on data from a motion sensor module) may indicate a user that is at risk. In such instances, an alert or other notification may be provided to the user or caregiver.

Further, a patient engaging in low-intensity ambulation (as determined by data from motion sensor module) who develops mouth breathing (whereas it was not present in prior days) indicate possible deteriorating disease and can serve as a trigger for further processing of the audio data, or provide another piece of input for processing (in combination with other inputs including lung sounds, chest wall movement, and inhaler use).

In another embodiment, the motion sensor module is used to monitor additional physiological parameters. For example, the motion sensor module can be used to monitor, for example, chest wall expansion, average tidal volume, respiratory rate, airflow rate, minute ventilation, and heart rate. These additional parameters can be important in evaluating patient health. For example, in some diseases tidal volume is a more reliable marker of pulmonary decompensation than respiratory rate.

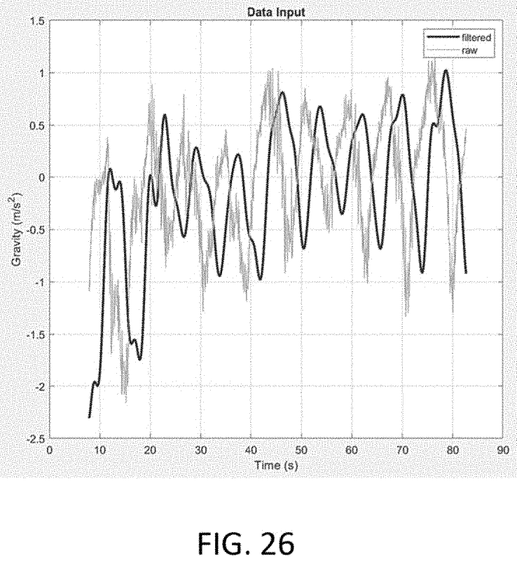

In one embodiment, the wearable device 1100 is positioned at the point of maximum impulse (PMI) (i.e., the position at which oscillatory motion of the chest due to heart beat is most prominent). Alternatively, the motion sensor module can be used to detect heart rate via ballistocardiography when the device is not placed near the PMI. As mentioned above, the motion sensor module can include one or more accelerometers, a magnetometer, and a gyroscope. The signal from each of these sensors can be converted to standard units (e.g., m/s2) and summed. A low pass filter is applied to the data. FIG. 26 shows exemplary raw summed data and the data after the low pass filter is applied.

Respiration information can be determined by analyzing the data captured by the motion sensor module. A double integration method may be used to translate the accelerometer data into position data. After the raw acceleration and time data from the device is filtered and processed to display the correct units, it is integrated using the trapezoidal method of integration once to determine the velocity, then a second time to get a position vector. This position vector is then evaluated to find the individual breath waveforms.

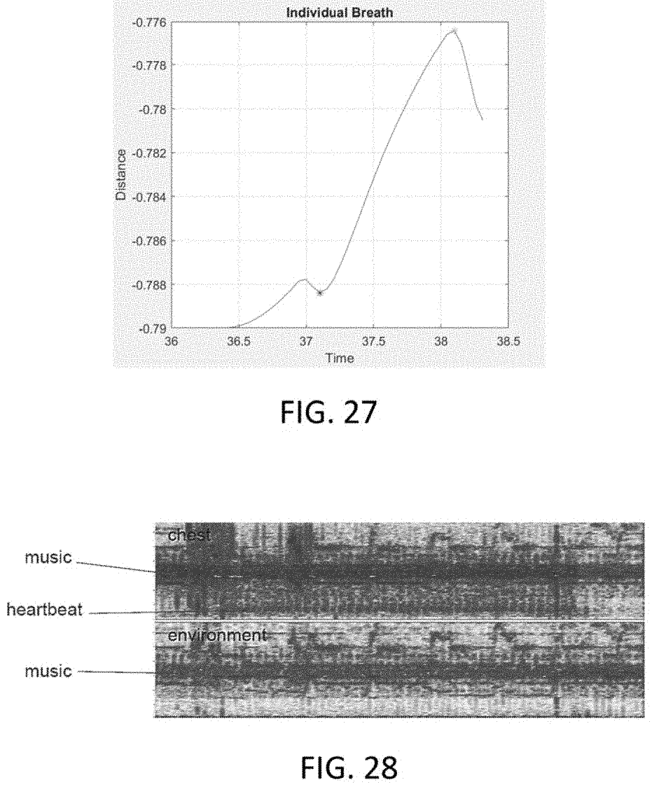

This position data can be used to determine tidal volume and chest wall expansion. For example, the data can be graphed. The peaks and valleys of the graphs correspond to the maximum volume and minimum volume, respectively, of the lungs. A peak locator function can used to locate the peaks. After identification of the peaks and valleys, the algorithm can split the data into separate breaths. The total distance traveled during each breath can then be calculated. An exemplary plot of a single breath is shown in FIG. 27.

The calculation of tidal volume can be further improved by using motion data captured by motion sensor module in conjunction with audio data received from microphones 1120, 1122. For example, the amplitude of chest wall movement can be used to calculate the tidal volume, as described herein. In some embodiments, the reliability of this determination may be assessed based on respiratory sounds captured by, for example microphones 1120, 1122. The correlation of chest wall motion with tidal volume may be based on the assumption that the patient's airways are patent. As a result, if the patient's airways are not patent, the calculation of tidal volume based on chest wall motion may be inaccurate. Patency of the airway can be assessed by respiratory sounds. For example, chest wall movement that correlates with a tidal volume of 550 cc may be classified as accurate when respiratory sounds are normal (as determined by audio data captured by microphones 1120, 1122). The same chest wall movement, when associated with wheezes (as determined by audio data captured by microphones 1120, 1122) may be classified as less accurate. Similarly, the same chest wall movement may be classified as inaccurate when associated with absent of breath sounds (as determined by audio data captured by microphones 1120, 1122).

Additionally, in one embodiment the loudness of respiratory sounds may be correlated with the amount of air flow in the respiratory system. From the amount of flow and the duration of respiratory sounds, the tidal volume may be estimated. In such embodiments, the determination based on audio data may be compared with the determination based on chest wall movement to verify and/or adjust the calculation of tidal volume.

In addition, in some embodiments, the user wears more than one wearable device 1100, allowing for more accurate calculation of the tidal volume. For example, in some embodiments, the user wears at least one device on each side of the user's torso. In some embodiments, one wearable device 1100 is positioned on the anterior/superior chest wall and a second wearable device 1100 is positioned on the xiphoid process of the user. The wearable device 1100 on the anterior/superior chest wall may be best positioned to capture chest wall movement. The wearable device 1100 positioned on the xiphoid process may be best positioned to capture different types of breathing styles, such as shallow breathing and belly breathing.

In some embodiments, the tidal volume (i.e., the amount of air that the patient moves in one minute) is also calculated based on the tidal volume and the rate of respiration. This may be done using both audio and motion data. A rapid increase or decrease in minute ventilation may indicate that the patient's condition is deteriorating and caregiver attention is required. In such instances, the wearable device 1100 may issue or transmit an alert.

A heart beat can be distinguished from respiration based on the frequency of the signal and the magnitude of the movement of the chest wall. These differences are used to filter the signal to distinguish heart rate and respiration. The heartbeat waveforms can be isolated by correlating the vector magnitude among the three different sensors in the motion sensor module. The comparison of the waveforms of the individual sensors can be compared to identify the heart beats.

In addition to measuring and/or calculating linear displacement of the chest wall, angular displacement can be measured and/or calculated as well. The angular displacement can be used in addition to or as alternative to the linear displacement. The angular displacement can be determined based on a gyroscope of the motion sensor module. The linear and/or angular velocity of the chest wall can also be used to determine the airflow rate.

Because the wearable device 1100 detects both physiological sounds as well as movement of the chest wall, the accuracy of the identification of abnormalities and/or patterns in breathing can be improved. For example, the combination of motion sensors and microphones can be used to identify individuals with diminished breath sounds, such as those suffering from severe bronchospasm. The motion sensor module can be used to identify phases in the respiratory cycle, as described above. Comparing the data gathered by the microphones during the various phases allows for more accurate identification of abnormalities in breath sounds.

Additionally, using the data from the motion sensor module in conjunction with the data from the microphone(s) 1120, 1122 may allow for the differentiation of wheeze and stridor. These two conditions result in similar respiratory sounds. However, these sounds occur at different phases of the respiratory cycle. Hence, it may be difficult to differentiate these conditions using sound alone. However, by comparing the timing of the respiratory sounds with the chest wall movement data gathered by the motion sensor module 317, these conditions can be identified.

In one embodiment, the data gathered by the wearable device 1100 is used to provide information regarding the patient during physical therapy. In such an embodiment, lung sound, chest wall motion, and other motion data including heart rate, posture, activity level, and gait are provided to the physical therapist or other caregiver via a software platform. Based on the data collected, real-time feedback and decision support is provided to the physical therapist for personalized therapy. Trending data can also be used to trend progress over time. This information can be used by the physical therapist to assess the patient's health and the efficacy of the physical training program. If necessary, the physical therapist can then make modifications to the training program. For example, if the patient's breathing is labored and/or abnormal, the physical therapist can reduce the intensity of the program. Alternatively, if the patient's breathing is within the desired range and is not indicative of an abnormality, the intensity of the program can be increased. The wearable device 1100 may also allow the patient to safely perform training routines when the physical therapist is not present by providing continuous monitoring of the patient's breathing, heart rate, and other metrics. A physical therapist or physician can review this information, either during the exercise or at a later time, to ensure that the patient is not in danger.

The wearable device 1100 can also be used to monitor compliance with prescribed or recommended activities. For example, incentive spirometry is often prescribed to prevent atelectasis in post-surgical patients. In one embodiment, the wearable device 1100 includes a user interface that provides real-time feedback and instructions on prescribed rehab activities based on sensor data. Concurrently, sensor data can be sent to family members and clinical providers to monitor compliance and progress.

The microphones 1120, 1122 can also be used to detect other physiological events. In one embodiment, the wearable device 1100 is placed on or near a major blood vessel. The wearable device 1100 can detect the sound associated with blood flow through the blood vessel. The sound of blood flow through a blood vessel can be used to monitor narrowing of blood vessels, or “stenosis” of blood vessels, changes in the state of surgical stents, and changes in blood flow. The wearable device 1100 can also detect the changes in the vibration of the skin surrounding the blood vessel, which correlates with the physiological state of the blood vessel wall, heart rate, and blood pressure, as well as the tissues that surround the blood vessel. Body sounds and motions then undergo processing by comparing the sounds to boundary conditions derived from predefined mathematical features derived from benchmark audio and motion data, as described above. This information can be used to diagnose or monitor vascular diseases, which include but are not limited to peripheral artery disease, carotid artery stenosis, abdominal aortic aneurysm, and access sites of endovascular procedures.

In another embodiment, the wearable device 1100 is placed on or near a joint of the patient (e.g., the shoulder, the elbow, the hip, the knee, the ankle). The acoustic sound generated by the joint during movement is used to monitor orthopedic diseases. In one embodiment, a wearable device 1100 is placed over more than one joint. For example, one wearable device can be placed over the left hip and one wearable device can be placed over the right hip. In such an embodiment, comparison of the data collected from the two devices allows for the identification of abnormalities in, for example, gait patterns. The identification can be performed by comparing the data collected to mathematical features derived from benchmark audio and motion data, as described above.

In another embodiment, the device is placed on the abdomen to detect abdominal sounds and abdominal movement. Acoustic analysis of abdominal sounds and the changes in abdominal movement undergo processing, as described above, to detect conditions that lead to fluids in the abdomen, rigidity of the abdominal wall, obstructions of the bowels, pseudo-obstructions of the bowels, and constipation.

In a further exemplary embodiment, the external computer (e.g., a smartphone, tablet computer, laptop computer, cloud-based computing system) modulates the frequency with which each sensor captures data.

The results of step 1218 can be displayed and/or arranged in numerous manners. For example, it is possible to perform classification of audio data with boundaries set by user input. The classification can also be performed based on sensor data (i.e. gyroscope) included in a smartphone.

In one exemplary embodiment, a patient is able to provide feedback—i.e. a self-assessment of the diagnosis, in order to improve the accuracy of diagnosis. Regardless, historical data can be accumulated over periods of time (days, months, years) to further refine boundary conditions and models used to identify respiratory problems.

In one exemplary embodiment, a computing device other than a smartphone may be used. Exemplary computing devices include computers, tablets, etc.

In one exemplary embodiment, results of identification of respiratory illness, and/or changes in respiratory conditions, are provided to a patient provider. The identification and/or changes may be displayed using a variety of different user interfaces. In one embodiment, wearable device 1100 provides an indication of remaining battery life.

In one exemplary embodiment, near-field communication (NFC) enabled tags are used to track medication and inhaler use. An NFC-enabled tag is attached to an inhaler or a medication container. After each use of the inhaler or each dose of medication, a user taps an NFC-enabled computing device to the NFC-enabled tag. The NFC-enabled computing device then records the time at which the tap occurs, which corresponds to the timing of the use of an inhaler or administering of a medication. The NFC-enabled computing device may include but is not limited to the following: mobile phone, tablet, or as part of the electronic components 1103. The output of medication-use tracking is a “boundary condition” described above.

In one exemplary embodiment, results of identification and/or changes are pushed to a patient or to a patient provider. In another exemplary embodiment, results of identification and/or changes are pulled to a patient or to a patient provider (i.e. provided on demand).

In one exemplary embodiment, results of identification and/or changes are provided to a patient and/or patient provider in the form of emails and/or text messages and/or other forms of electronic communication. In one exemplary embodiment, the results are displayed in a software application (“app”) operating on a smartphone or other computing device.

The sampling frequency and sampling duration set forth above are merely exemplary. In one exemplary form of the present invention, sampling frequency and/or duration may be changed.

In one exemplary embodiment, the invention is used in combination with location technology such as GPS in order to locate location of a patient.

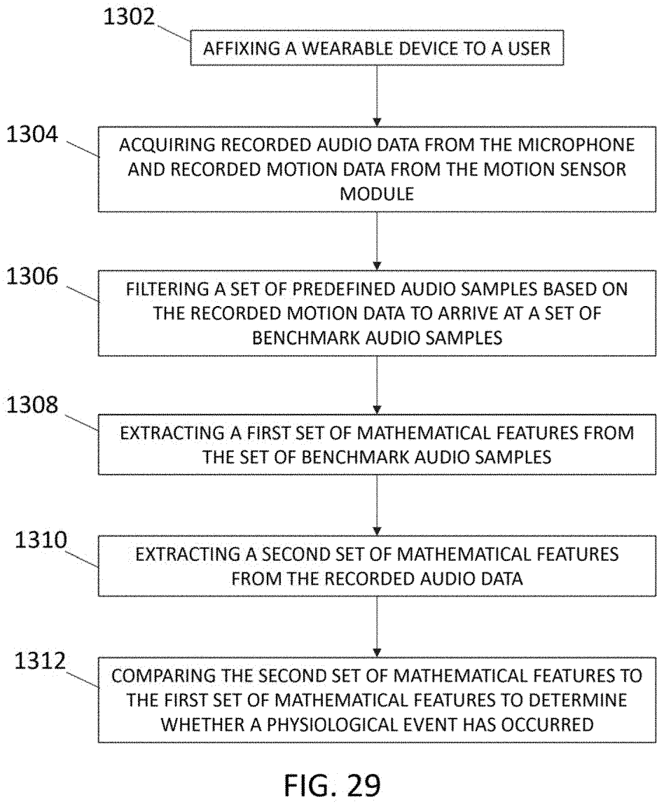

In one embodiment, shown in FIG. 29, a method of identifying physiological events is provided. The method includes affixing a wearable device to a user (step 1302). The wearable device includes at least one microphone, a motion sensor module, and a processor. The method further includes acquiring recorded audio data from the at least one microphone and recorded motion data from the motion sensor module (step 1304) (e.g., physiological data). The method further includes filtering a set of predefined audio samples based on the recorded motion data to arrive at a set of benchmark audio samples (step 1306). The method further includes extracting a first set of mathematical features from the set of benchmark audio samples (step 1308). The method further includes extracting a second set of mathematical features from the recorded audio data (step 1310). The method further includes comparing the second set of mathematical features to the first set of mathematical features to determine whether a physiological event has occurred (step 1312). In some embodiments, steps 1306-1312 are performed by one or more trained AI-models, as previously discussed.

In one embodiment, the set of predefined audio samples are recorded from multiple subjects.

In one embodiment, the method further comprises, when the comparing step determines that a physiological event has occurred, performing a verification of the determination based on a comparison of additional mathematical features extracted from the recorded audio data with additional mathematical features extracted from the benchmark audio samples.

In one embodiment, the at least one microphone includes a first microphone and a second microphone, the first microphone oriented toward the user and the second microphone oriented away from the user. In such an embodiment, the method further includes subtracting the signal from the second microphone from the signal generated by the first microphone prior to extracting the second set of mathematical features.

In one embodiment, the filtering step further includes filtering the predefined spectrograms based on user data. In such an embodiment, the user data is selected from the group consisting of surgical history, disease condition, medication use, demographics, user weight, and user height.

In one embodiment, the wearable device is affixed at the point of maximum impulse.

In one embodiment, the wearable device is affixed adjacent a joint of the user.

In one embodiment, the wearable device is affixed to the abdomen of the patient.

In one embodiment, the method further includes exporting the recorded audio data and the recorded motion data to a computing device and analyzing the recorded audio data and the recorded motion data using the computing device to verify the determination of whether the physiological event has occurred. In one such embodiment, the analyzing step includes analyzing the recorded audio data and the recorded motion data based at least partially on parameters not used in the comparing step.

In another aspect, a system for providing feedback on physiological events is provided. The system includes a wearable device and a computing device. The wearable device is configured to be worn by a patient and includes at least one microphone configured to capture recorded audio data. The wearable device also includes a motion sensor module configured to capture recorded motion data. The wearable device also includes a processor configured to determine whether a physiological event has occurred based on the recorded audio data and the recorded motion data and generate a signal when the physiological event has occurred. The computing device includes a display and is in communication with the wearable device. The computing device is configured to: (i) receive the recorded audio data from the wearable device; (ii) receive the recorded motion data from the wearable device; (iii) receive the signal from the processor; and (iv) provide a graphical user interface on the display indicating that the physiological event has occurred.

In one embodiment, the computing device is a smartphone. In another embodiment, the computing device further includes a processor, the processor configured to analyze the recorded audio data and the recorded motion data based at least partially on parameters not used by the processor of the wearable device.

In another aspect, a non-transitory computer readable medium containing computer-executable programming instructions for performing a method of identifying physiological events is provided. The method includes acquiring recorded audio data from at least one microphone and recorded motion data from a motion sensor module, the at least one microphone and the motion sensor module being housed in a wearable device affixed to a user. The method also includes filtering a set of predefined audio samples based on the recorded motion data to arrive at a set of benchmark audio samples. The method also includes extracting a first set of mathematical features from the set of benchmark audio samples. The method also includes extracting a second set of mathematical features from the recorded audio data. The method also includes comparing the second set of mathematical features to the first set of mathematical features to determine whether a physiological event has occurred. The method also includes causing a graphical user interface to responsively display an indication that the physiological event has occurred.

In another aspect, a method for analyzing respiratory motion is provided. The method includes affixing a wearable device to a user. The wearable device includes a motion sensor module. The method further includes acquiring recorded motion data from the motion sensor module. The method further includes calculating the movement of the chest wall to determine tidal volume of a respiration cycle.

In another embodiment, the wearable device includes at least one microphone and the method further includes acquiring recorded audio data with the at least one microphone, the recorded audio data including respiratory sounds. The method also includes determining the phase of the respiratory cycle during which the respiratory sounds occur based on the recorded motion data.

In another aspect, a method of identifying physiological events is provided. The method includes affixing a wearable device to a user. The wearable device includes at least one microphone and a processor. The method further includes acquiring recorded audio data from the at least one microphone. The method further includes filtering a set of predefined audio samples based on user data to arrive at a set of benchmark audio samples. The method further includes extracting a first set of mathematical features from the set of benchmark audio samples. The method further includes extracting a second set of mathematical features from the recorded audio data. The method further includes comparing the second set of mathematical features to the first set of mathematical features to determine whether a physiological event has occurred.

In one embodiment, the user data is selected from the group consisting of surgical history, disease condition, medication use, demographics, user weight, and user height.

In another aspect, a method of identifying physiological events is provided. The method includes affixing a wearable device to a user. The wearable device includes at least one microphone and a processor. The method further includes acquiring recorded audio data from the at least one microphone. The method further includes extracting a first set of mathematical features from a set of benchmark audio samples. The method further includes applying an adjustment to the first set of mathematical features to determine adjusted mathematical features. The method further includes extracting a second set of mathematical features from the recorded audio data. The method further includes comparing the second set of mathematical features to the adjusted mathematical features to determine whether a physiological event has occurred.

In one embodiment, the wearable device includes a motion sensor module and the method includes acquiring recorded motion data from the motion sensor module. The method further includes using the recorded motion data to calculate the adjusted mathematical features.

In one embodiment, the adjusted mathematical features are calculated using user data. The user data selected from the group consisting of surgical history, disease condition, medication use, demographics, user weight, and user height.

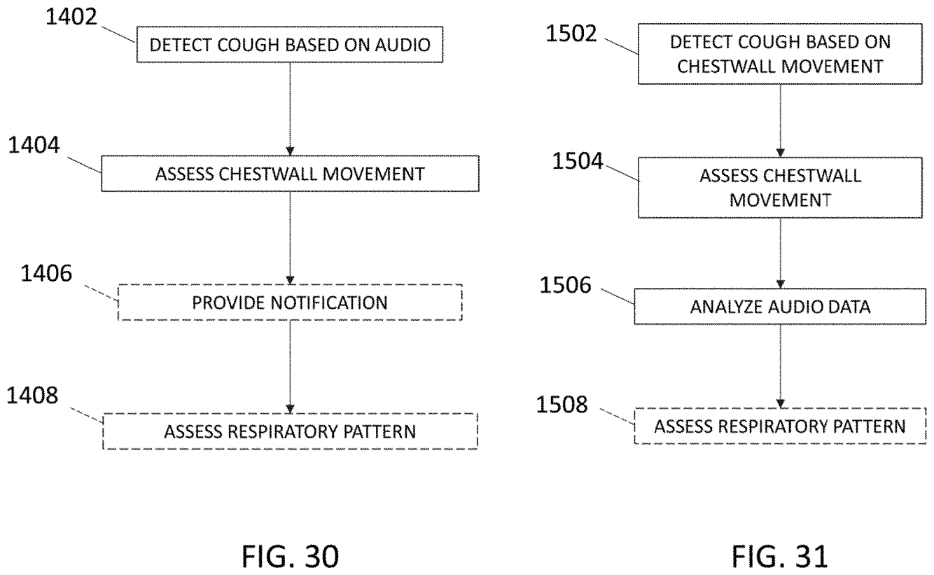

FIGS. 30 and 31 show methods of determining the aspiration risk associated with a cough detected using data gathered by wearable device 1100. In FIG. 30, at step 1402, the cough is first detected based on audio using microphone 1120 and/or microphone 1122. The cough may be identified using any of the processes described herein. After identifying the cough, at step 1404, the user's chest wall movement is assessed. This assessment may be based on data received from motion sensor module. If the user's chest wall movement does not reflect that the user coughed, it may be determined that the user did not actually cough. For example, someone else in the area may have coughed or other ambient noises may have created the cough-indicative audio data. If, on the other hand, the motion data indicates that the chest wall did experience movement indicative of a cough, assessing the amplitude and/or acceleration of the chest wall movement. This may allow for a determination of whether the cough was a strong cough or a weak cough. For example, a high amplitude and/or acceleration of movement of the chest wall may indicate that it was a strong cough, with a corresponding low aspiration risk. On the other hand, a low amplitude and/or acceleration of chest wall movement may indicate a weak cough, with a corresponding higher aspiration risk. At step 1408, the respiratory pattern of the user may be assessed, based on motion data, to determine when in the respiratory cycle the cough occurred. This may further allow for a determination of the aspiration risk.

Turning to FIG. 31, at step 1502, the cough may be detected based on chest wall movement using motion data received from motion sensor module. For example, the cough may be identified by analysis of chest wall motion, velocity, acceleration, and derivatives thereof. After detecting a cough, at step 1504, the chest wall movement data may be assessed to determine if the cough was a strong cough or a weak cough. At step 1506, the audio data received from microphone 1120 and/or microphone 1122 may be analyzed. For example, if the analysis of the chest wall movement indicates that a strong cough has occurred, and the analysis of the audio data confirms this, it may be determined that a strong cough, with a low aspiration risk, has occurred. On the other hand, if the analysis of the chest wall movement indicates a strong cough, but the analysis of the audio data does not confirm a strong cough, this may indicate an obstruction of the user's upper airway. In such a scenario, the wearable device 1100 may issue a notification to the user or a caregiver to check for an upper airway obstruction.

If the analysis of the chest wall movement indicates a weak cough and the audio data indicates a strong cough, this may be indicative of an error. For example, the wearable device 1100 may be incorrectly positioned on the user's chest wall. If, instead, the analysis of the chest wall movement indicates a weak cough and the analysis of the audio data confirms this assessment, it may be determined that a weak cough has occurred. As described above, optionally, the respiratory pattern of the user may be assessed, based on motion data, to determine when in the respiratory cycle the cough occurred. This may further allow for a determination of the aspiration risk.

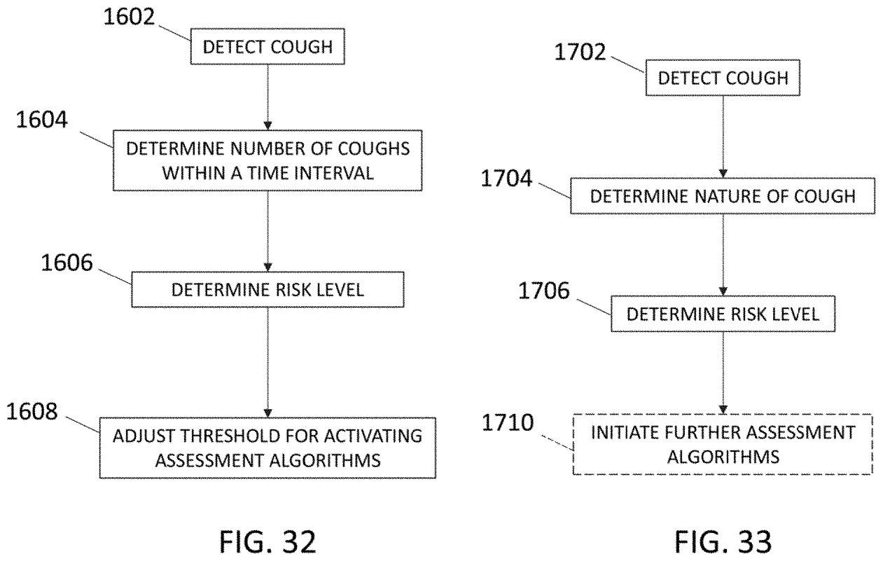

A method of determining the risk associated with a cough is shown in FIG. 32. At step 1602, a cough is detected. The cough may be detected through any of the processes described herein. For example, the cough can be detected by analyzing audio data received from microphone 1120, 1122 or additional physiological data (such as motion data) received from the multi-sensor module 1128. At step 1604, the number of coughs occurring within a given interval is determined to identify clusters of coughs. For example, a cluster may be identified when three or more coughs are identified within 30 seconds. In other embodiments, different numbers of coughs or different durations (e.g., 10 seconds, 5 minutes, etc.) may be used to classify cough clusters. In some embodiments, the motion data can be used to identify cough clusters where an audio-based approach only identifies a single cough (i.e., when the patient's glottis is closed during a cough, or a loud ambient sound masks additional coughs). At step 1606, based on the frequency of the coughs, a risk level associated with the coughs is determined. At step 1608, based on this risk level, the threshold for activating further assessment algorithms may be adjusted. Assessing the risk in this way has a number of advantages. For example, by only implementing further assessment when a high-risk cluster of coughs is identified, battery and computing power may be conserved.

FIG. 33 illustrates a method of determining cough characteristics. At step 1702, a cough is detected. The cough may be detected through any of the processes described herein. For example, the cough can be detected by analyzing audio data received from microphone 1120, 1122 or additional physiological data (such as motion data) received from the multi-sensor module 1128. At step 1704, the nature of the cough is determined (e.g., whether the cough is a dry cough or a wet cough). This may be done based on audio data received from microphone 1120, 1122, for example. In some embodiments, motion data received from motion sensor module is used to determine whether the cough was a “strong” cough or a “weak” cough. Based on the nature and characteristics of the cough, an aspiration risk level may be determined. For example, a dry cough has a relatively lower risk of infection and/or aspiration, while a wet cough has a relatively higher risk of infection and/or aspiration. Based on the determination of the level of risk, at step 1710, further assessment algorithms may be initiated. By only initiating further assessment algorithms when a high-risk cough is detected, computing and battery resources may be conserved.

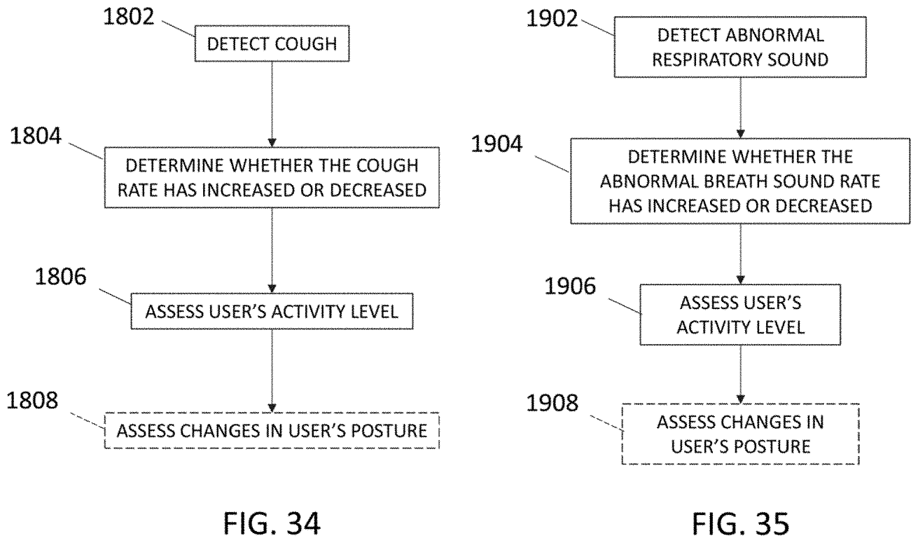

FIG. 34 illustrates another method of identifying a risk level associated with a cough. At step 1802, a cough is detected. The cough may be detected through any of the processes described herein. For example, the cough can be detected by analyzing audio data received from microphone 1120, 1122 and/or additional physiological data (such as motion data) received from the multi-sensor module 1128. At step 1804, a determination is made of whether the cough rate has increased or decreased. For example, the number of coughs identified in the previous 24 hours may be compared with those received in the prior 72 hours. In addition, at step 1806, the user's activity level may be assessed based on motion data received from the multi-sensor module 1128. If the rate of coughs has increased and the user's activity level has increased as well, the increased cough rate may be a result of exercised induced bronchospasm. In such a situation, no further action may be required. Further, if the cough rate has decreased and the activity level has increased, this may be an indication of improving symptoms. A decrease in cough rate and coincident decrease in activity level may indicate that there has not been a significant change in the user's symptoms.

In some embodiments, at step 1808, changes in the user's posture may be assessed using motion data received from multi-sensor module 1128. This may further assist with assessment of the user's condition. For example, if the user's cough rate has increased, the user's activity level has remained substantially the same or decreased, and the user's posture indicates that the user is lying down, this may indicate that the user is experiencing night time symptoms. In some instances, this may also indicate that the user is experiencing worsening heart failure. In instances in which the user's cough rate has increased, the user's activity level has remained the same or decreased, and the motion data indicates that the user is not lying down, this may be an indication that the user's symptoms are worsening. In some instances, this may also indicate that the user is experiencing worsening heart failure.

On the other hand, in instances in which the user's cough rate is decreasing, the user's activity level has remained substantially the same, and the user's posture has not changed, this may indicate that the user's symptoms are improving. In instances in which the user's cough rate is decreasing, the user's activity level has remained substantially the same, and the user's posture has not changed, this may indicate that the change in cough rate is posture related.

FIG. 35 illustrates a method for assessing the risk associated with an abnormal respiratory sound. The method includes many of the same processes and assessment as those described above with respect to FIG. 34. At step 1902, an abnormal respiratory sound may be detected. For example, the abnormal respiratory sound may be detected based on audio data received from microphone 1120, 1122. The abnormal respiratory sound may include, but is not limited to, a wheeze or rhonchi. At step 1904, it may be determined whether the rate at which the abnormal breath sound is occurring has increased or decreased. For example, the number of abnormal respiratory sounds identified in the previous 24 hours may be compared with those identified in the prior 72 hours. At step 1906, the user's activity level may be assessed based on motion data received from multi-sensor module 1128. Optionally, at step 1908, changes in the user's posture may be assessed based on motion data received from multi-sensor module 1128. Based on this information regarding the rate of abnormal respiratory sounds, the user's activity level, and changes in the user's posture, a risk level may be determined as described above with reference to FIG. 34. For example, in instances in which the rate of abnormal respiratory sounds has increased and the user's activity level has increased, this may indicate that the increased abnormal respiratory sound rate is related to exercise induced bronchospasm.

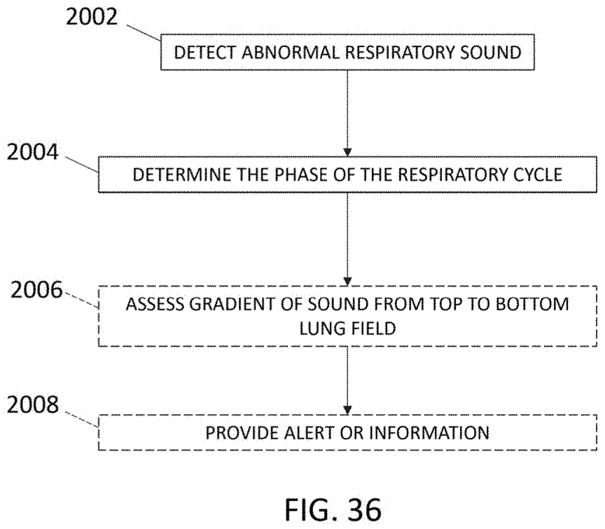

FIG. 36 illustrates another method of characterizing abnormal respiratory sounds, such as adventitious breath sounds. This may include, for example, wheezes, rhonchi, and rales. At step 2002, an abnormal respiratory sound may be detected using audio data received from microphone 1120, 1122. At step 2004, the phase of the respiratory cycle in which the abnormal respiratory sound occurred may be determined using motion data received from multi-sensor module 1128. In instances in which the abnormal respiratory sound occurs during the expiratory phase or both the expiratory and inspiratory phase of the respiratory cycle, the level of risk may be relatively low and information to be reviewed by a clinician may be generated, at step 2008.

On the other hand, if the user is wearing multiple devices (e.g., a first device and a second device) in instances in which the abnormal respiratory sound occurs during the inspiratory phase, it may be determined, at step 2006, whether there is a gradient between the upper and lower lung field. If there is no such gradient, or the gradient is low, the risk level may be relatively low, and information may be generated for a clinician to review, at step 2008. On the other hand, if there is a significant gradient between the upper and lower lung fields, this may indicate that the user has experienced a stridor. In such instances, an alert may be generated to make the user or a caregiver aware of the risk. The alert may be, for example, an audible alert or a tactile alert (e.g., vibration). Alternatively, or additionally, a text message, email, or other text-based alert may be generated and transmitted to the user, a caregiver, or a clinician.

In some instances, the abnormal respiratory sound identified using the audio data is an adventitious breath sound (e.g., wheezes, rhonchi, whistles, etc.). In other instances, the abnormal respiratory sound is indicative of the user's use of an inhaler. In such instances, the audio data can be used to determine the type of inhaler being used. This may be done using audio data received from the chest facing microphone 1120 as well as the background microphone 1122. Different types of inhalers lead to different types of sounds that can be identified in the audio data. Further, the audio data can be analyzed to identify lung sounds occurring during inhaler use. Further, the motion data can be analyzed to determine in which phase of the respiratory cycle the inhaler is used (e.g., based on chest wall movement).

The analysis of the user's use of the inhaler may be used to identify incorrect inhaler use. Many patients employ the wrong technique when using their inhalers, leading to suboptimal dosage. Deviation from normal inhaler sound and chest wall movement can be used to identify inhaler misuse. Specifically, the timing of inhaler “clicks” and/or the timing of respiratory sounds indicative of inhaler use as compared to chest wall movements, could be used to identify inhaler misuse.

Although the subject matter has been described in terms of embodiments, the claims should be construed broadly, to include other variants and embodiments, which may be made by those skilled in the art.

What is claimed is:

1. A system, comprising:
a memory having instructions stored thereon, and a processor configured to read the instructions to:
receive a training data set comprising physiological data including labeled events corresponding to a predetermined portion of the physiological data;
generate a trained artificial intelligence (AI) model configured to identify events within device data, wherein the trained AI model is generated using an iterative training process based on the training data set; and
identify at least one physiological event within a target device data set based on the trained AI model.

2. The system of claim 1, wherein the trained AI model is configured to clean the device data prior to marking events within the device data, and wherein the at least one respiratory event is identified by cleaning the device data to remove one or more artifacts.

3. The system of claim 1, wherein the training data set comprises one or more user preferences generated by interacting with a second trained AI model.

4. The system of claim 3, wherein the second AI model is generated based on a training data set without the one or more user preferences.

5. The system of claim 1, wherein the target device data set comprises physiological data.

6. The system of claim 4, wherein the physiological data is obtained by a wearable device.

7. The system of claim 1, wherein the training data set includes environmental data, and wherein the target device data set comprises environmental data.

8. The system of claim 7, wherein the environmental data comprises speech data, and wherein the trained AI model is configured to remove or mask the speech data.

9. The system of claim 7, wherein the speech data is identified at least partially based on signal characteristics of the speech data.

10. The system of claim 1, wherein the trained AI model is generated using transfer learning techniques.

11. The system of claim 1, wherein the trained AI model is trained to interpret marked events.

12. The system of claim 1, wherein the trained AI model is trained to differentiate data originating from a first source associated with a device configured to obtain the target device data set and data originating from a second source not associated with the device.

13. The system of claim 1, wherein the trained AI model is trained to validate generated markings.

14. The system of claim 1, wherein training data set includes metadata associated with at least one labeled event, and wherein the target device data set comprises metadata associated with at least a portion of the target device data.

15. An artificial intelligence (AI)-enabled environment, comprising:
a first staged processing layer configured to receive device data, wherein the first staged processing layer includes a trained AI model configured to identify at least one physiological event within the device data, wherein the trained AI model is generated based on a training data set comprising physiological data including labeled events corresponding to a predetermined portion of the physiological data;
a second staged processing layer, wherein the second staged processing layer is configured to receive first modified device data comprising a portion of the device data; and
at least one non-transitory storage configured to store at least one of the device data and the modified device data.

16. The AI-enabled environment of claim 15, wherein the first modified device data is generated by removing or masking speech data within the device data.

17. The AI-enabled environment of claim 15, wherein the first modified device data is generated by filtering the device data to include only data relevant to a predetermined use case.

18. The AI-enabled environment of claim 15, wherein at least one physiological event is identified within the first modified device data at the second staged processing layer.

19. The AI-enabled environment of claim 15, comprising a user interface generated by a second trained AI model, wherein the second trained AI model is generated using a training data set comprising user preferences.

20. A computer-implemented method of processing device data, comprising:
receiving device data from a first device;
cleaning the device data to remove at least one artifact using a trained artificial intelligence (AI) model, wherein the trained AI model is generated based on a training data set comprising physiological data including labeled events corresponding to a predetermined portion of the physiological data;
marking the device data to identify at least one physiological event using the trained AI model; and
outputting the cleaned and marked device data for use in a AI training process configured to train a second trained AI model to identify physiological events.
